(19) United States
(12) Patent Application Publication
(10) Pub. No.: US 2009/0258041 A1
(43) Pub. Date: Oct. 15, 2009

(54) Protection of Oxidizable Agents

(76) Inventors: Sebastien Mongiat, Sierentz (FR); Werner Baschong, Basel (CH); Bernd Herzog, Grenzach-Wyhlen (DE)

Correspondence Address: JoAnn Villamizar;Ciba Corporation/Patent Department, 540 White Plains Road, P.O. Box 2005, Tarrytown, NY, 10591, US

(21) Appl. No.: 12/083,557

(22) PCT Filed: Jun. 21, 2006
(86) PCT No.: PCT/EP2006/063421

§ 371 Date: Apr. 14, 2008

(30) Foreign Application Priority Data
Oct. 24, 2005 (EP) PCT/EP2005/055475

(51) Int. Cl. A61K 8/02; A61P 17/02

(52) U.S. Cl. 424/401

(57) ABSTRACT

Formulations for cosmetical or pharmaceutical use contain an antioxidant in encapsulated form and a cosmetically or pharmaceutically acceptable carrier. The antioxidant is selected from carbon bridged hindered phenoles, ester bridged hindered phenoles, amide bridged hindered phenoles, lactones of hindered phenoles, sterically hindered oxylamines and sterically hindered hydroxylamines. The encapsulated antioxidant is of high activity and suitable to protect a further active ingredient, e.g. oxidizable natural substances, vitamins, fragrances, and extracts from plants fungi, algae or animals, from premature degradation, especially when coencapsulated together with the ingredient. The formulations are useful inter alia for the preparation of a cosmetical or pharmaceutical skin care or skin delivery formulation, injectable solution, infusion solution, eye drop, drinking solution, a dietary or enriched food, an oral care formulation, drinking gargles, in-halants, or as a food additive.

The present invention relates to the use of encapsulated forms of certain antioxidants, especially for the protection and stabilization of active ingredients in a cosmetical or pharmaceutical formulation. The protection is most effectively achieved by coencapsulation together with the active agent. The invention further relates to corresponding cosmetical or pharmaceutical compositions, and to the use for the preparation of medicaments or formulations for the treatment of radical induced impairments such as inflammatory or allergic conditions, collagen damages, DNA-damage, or reperfusion-damage (use as anti aging).

The use of certain phenolic antioxidants or amino compounds for the stabilization of cosmetics or household formulations has been proposed in WO 00/25731 and WO 03/103622. IPCOM000130489D (see e.g. under https://priorart.ip.com/viewPub.isp) inter alia suggests using such antioxidants for the protection of oxygen-sensitive components of the formulation.

### SUMMARY OF THE INVENTION

It has now been found that certain antioxidants are especially useful when contained in a cosmetical or pharmaceutical formulation in encapsulated form. These antioxidants, which are selected from carbon or ester/amide bridged hindered phenoles, lactones of hindered phenoles, sterically hindered oxylamines and sterically hindered hydroxylamines, inter alia are suitable to protect other active ingredients, such as oxidizable natural substances or active ingredients such as vitamins, plant extracts, fragrances etc., from premature degradation or hydrolization, e.g. induced by light, oxygen and/or heat, especially when these antioxidants are encapsulated together with the ingredient (coencapsulation). Formulations may be purely cosmetical ones, or formulations making use of the pharmacological advantages of the active ingredient as well as the antioxidant. The formulations usually comprise at least one lipid phase and at least one water phase, preferably in the form of a microemulsion, microdispersion or especially nanoemulsion or nanodispersion. The present antioxidants further exhibit marked radical scavenging and antiinflammatory action in cellular and enzymatic in vitro assays and in in vivo assays on human volunteers, while showing good skin and cell compatibility. Damage of radicals in the organism of animals and humans, which may be prevented by the present antioxidants, has been described in further detail in U.S. Pat. No. 4,698,360. The use of the antioxidant is most advantageous for the protection of a vitamin such as vitamin A, E or C or modified form thereof.

### DETAILED DESCRIPTION OF THE INVENTION

Hindered phenoles are a group of antioxidants comprising active aromatic compounds containing at least one hydroxy substituent on an aromatic ring and one or two further aliphatic or aromatic substituents, often an alkyl group such as methyl or isopropyl or tert.-butyl or tert.-amyl, in ortho-position relative to said hydroxy substituent. Carbon bridged hindered phenoles contain at least one carbon bridging group, ester bridged hindered phenoles contain at least one ester (—COO— or —OCO—) bridging group, amide bridged hindered phenoles contain at least one amide (—CONR— or —NRCO—, where R usually is a carbon residue or hydrogen) bridging group. The bridging group(s) either link(s) 2 or more phenolic moieties together to form one single compound, or link(s) one phenolic moiety with an anchor group (such as an alkyl chain or an aromatic ring). In lactones of hindered phenoles, the (phenolic) hydroxy substituent has been esterified to form an annealed ring. Sterically hindered oxylamines and sterically hindered hydroxylamines each are tertiary amines containing 2 residues which are branched in alpha-position; as 3^{rd} residue, the oxylamine contains an oxygen atom (thus being a radical), the hydroxylamine contains a hydroxy group.

Suitable antioxidants include compounds of the formulae

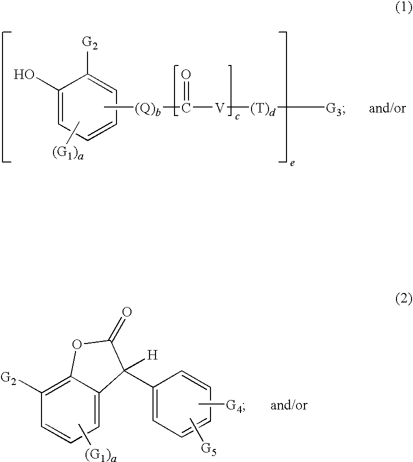

(3) hindered nitroxyl compounds, hindered hydroxylamine compounds, hindered hydroxylamine salt compounds of the formulae

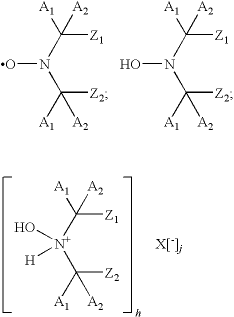

where in the above formulae
G₁ is hydrogen; C₁-C₂₂alkyl; C₁-C₂₂alkylthio; C₂-C₂₂alkylthioalkyl; C₅-C₇cycloalkyl; phenyl; C₇-C₉-phenylalkyl; or SO₃M;
G₂ is C₁-C₂₂alkyl; C₅-C₇cycloalkyl; phenyl; or C₇-C₉-phenylalkyl;
Q is —CₘH₂ₘ—;

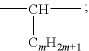

—CₘH₂ₘ—NH; a radical of formula

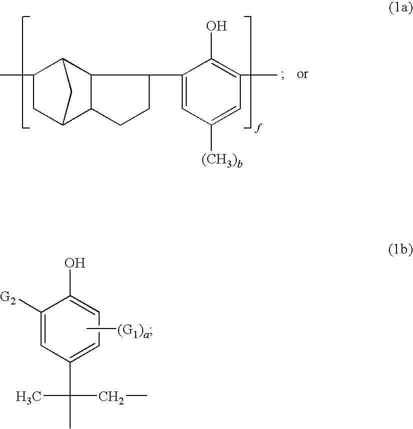

T is —CₙH₂ₙ—; —(CH₂)ₙ—O—CH₂—; phenylene;

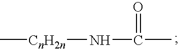

or a radical of formula

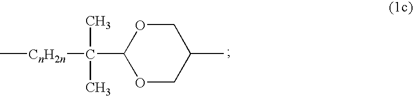

V is —O—; or —NH—;
a is 0; 1; or 2;
b, c and d and g are each independently of one another 0; or 1;
e is an integer from 1 to 4;
f is an integer from 1 to 3; and
m, n and p are each independently of one another an integer from 1 to 3;
q is 0 or an integer from 1 to 3;
if e=1, or in formula (3), then
G₃ is hydrogen; C₁-C₂₂alkyl; C₅-C₇cycloalkyl; C₁-C₂₂alkylthio; C₂-C₂₂alkylthioalkyl; C₂-C₁₈alkenyl; C₁-C₁₈-phenylalkyl; M; SO₃M; a radical of formula

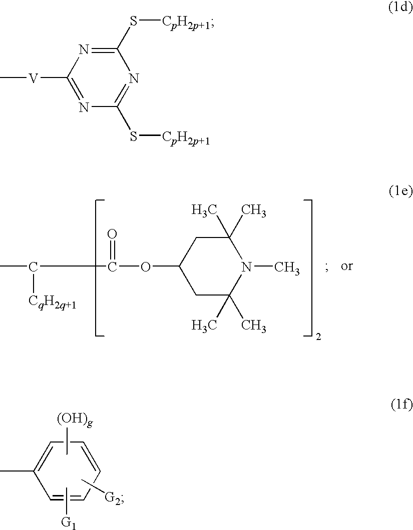

or G₃ is propyl substituted by OH and/or by C₂-C₂₂alkanoyloxy;
M is alkali; ammonium; H;
if e=2, then
G₃ is a direct bond; —CH₂—;

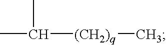

or —S—; or G₃ is propyl substituted by OH or C₂-C₂₂alkanoyloxy;
if
e=3, then
G₃ is the radical of formula (1g);

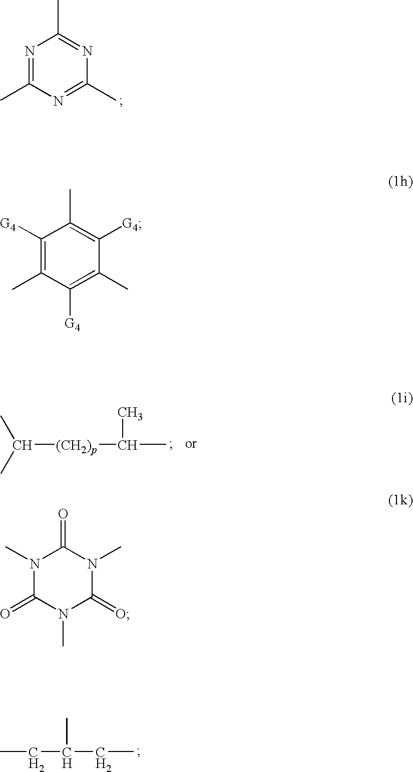

if
e=4, then
G₃ is

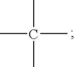

G₄ and G₅ are each independently of the other hydrogen; or C₁-C₂₂alkyl;
A₁ and A₂ are independently alkyl of 1 to 4 carbon atoms or are together pentamethylene,
Z₁ and Z₂ are each methyl, or Z₁ and Z₂ together form a linking moiety which may additionally be substituted by an ester, ether, hydroxy, oxo, cyanohydrin, amide, amino, carboxy or urethane group,
h is the number of positive charges and j is the number of negative charges,
X is an inorganic or organic anion, and
where the total charge of cations h is equal to the total charge of anions j.

Compounds of the formula 1, where e is 1, usually contain the spacer groups Q and (CO)—V, which corresponds to the condition b=c=1. Also useful are the compounds of formulae

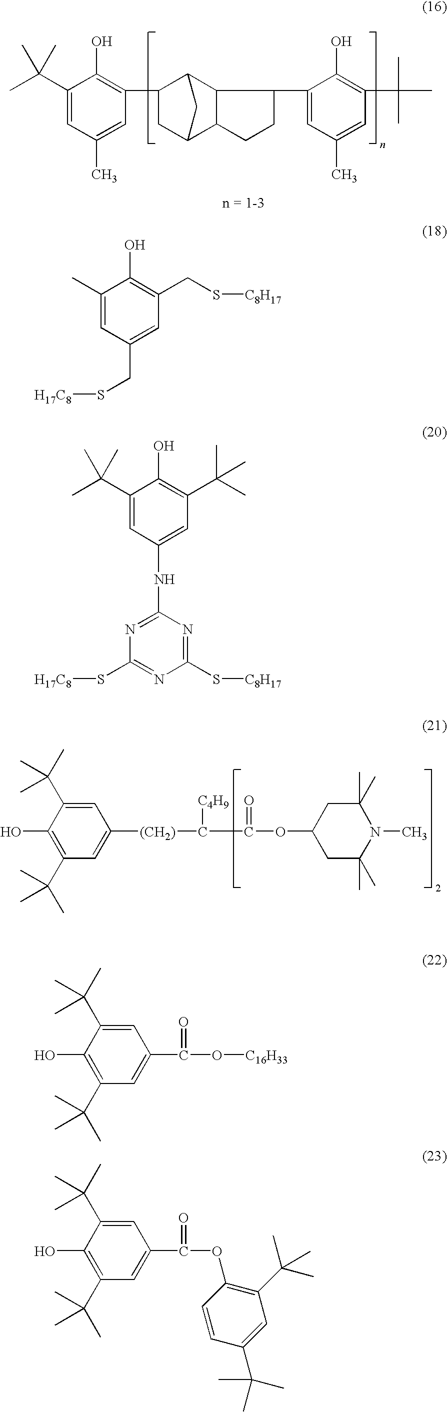

Encapsulation has been found to enhance the action of the antioxidant, especially where small particle sizes are achieved (e.g. size of the loaded particle in the nanoscale range of diameters lower than 300 nm or even lower than 100 or 50 nm, see further below). Present compounds can be used alone or as mixtures with each other and/or further components, e.g. those described further below. Present invention therefore includes a method for the protection of an active ingredient in a cosmetical or pharmaceutical formulation against premature degradation, e.g. by light, oxygen and/or heat, characterized in that at least one antioxidant is encapsulated, especially together with the ingredient. The active ingredient often is selected from oxidizable natural substances, vitamins, plant extracts, fragrances, especially the vitamins A, C, E. Though some stabilized forms of vitamins (hereinafter recalled as “modified forms”) have already been developed, e.g. vitamin esters or vitamin ester salts such as vitamin A palmitate, vitamin C magnesium phosphate (ascorbyl 2-phosphate magnesium salt), sodium ascorbyl 2-phosphate 6-palmitate, vitamin E acetate, there is still need for improvement of the stabilization of the free substances as well as of these modified forms, which is advantageously effected by the present invention.

Active ingredients to be stabilized according to the invention further include the group consisting of
tocopherol (α, β, γ, δ isomers) and its esters of acids with general formulas

H(CH2)n(CHR)COOH (1)

CH3(CH2)mCH═CH(CH2)nCOOH (2)

where R is hydrogen atom or OH group, m, n are integral numbers from 0 to 22 where m+n sum is maximally 22;
tocotrienol (α, β, γ, δ isomers), containing one unsaturated fatty chain, and its esters of acids;
ascorbic acid and its esters of acids such as phosphoric acid and also sodium, potassium, lithium and magnesium salts, Ascorbyl Tetraisopalmitate, further ester with pyrrolidoncarboxylic acid and esters of acids with general formulas

H(CH2)n(CHR)COOH (3)

CH3(CH2)mCH═CH(CH2)nCOOH (4)

where R is hydrogen atom or OH group, m, n are integral numbers from 0 to 20 where m+n sum is maximally 21;
retinoids including all natural and/or synthetic analogs of vitamin A or retinal-like compounds which possess the biological activity of vitamin A in the skin as well as the geometric isomers and stereoisomers of these compounds. Preferred compounds are retinal, retinol esters (e.g., C2-C22 alkyl esters (saturated or unsaturated alkyl chains) of retinal, including retinyl palmitate, retinyl acetate, retinyl propionate), retinal, and/or retinoic acid (including all trans retinoic acid and/or 13-cis-retinoic acid) or derivatives. Other retinoids which are useful herein are described in U.S. Pat. Nos. 4,677,120, issued Jun. 30, 1987 to Parish et al; 4,885,311, issued Dec. 5, 1989 to Parish et al; 5,049,584, issued Sep. 17, 1991 to Purcell et al., 5,124,356, issued Jun. 23, 1992 to Purcell et al. Other suitable retinoids are tocopheryl-retinoate [tocopherol ester of retinoic acid (trans or cis)], adapalene [6-(3-(1-adamantyl)-4-methoxyphenyl)-2-naphtoic acid] and tazarotene (ethyl 6-[2-(4,4-dimethylthiochroman-6-yl)-ethynyl]nicotinate);
carotenoids such as α-, β-, γ-, and δ-carotene, lutein, xanthophylls, zeaxanthine, violaxanthine, cryptoxanthine, fukoxanthine, antheraxanthine, lycopene, didehydrolycopene and tetradehydrolycopene carotenoids
enzymatic antioxidants such as Glutathione peroxidase, Catalase, Superoxide dismutase;
Ubiquinone and ldebenone (hydroxydecyl Ubiquinone), Ubiquinol and its derivatives;
lipoic acid and its derivatives such as alpha-lipoic acid;
rutinic acid and its derivatives such as α-glucosylrutin, a water soluble flavonoid, rutin hydrate (vitamin P);
plant extracts such as white and green tea extracts, chicory leaf extract (Cichorium intubybus), Passionflower extract (Passiflora incarnata), Aspalathus linearis extract, rosmary extract, red leaf extract of Aceraceae Maple tree or of Rosaceae Cherry tree, Curcuma longa L (curcuminoids active ingredients), Leontopodium alpinum extract, Emblica officinalis (phyllanthus emblica) tree extract;
phenolic acids such as caffeic acid, 3,4-dihydroxyphenyl acetic acid, 3,4-dihydroxybenzoic acid;
flavonoids and polyphenols such as flavanones selected from the group consisting of unsubstituted flavanones, mono-substituted flavanones, and mixtures thereof; chalcones selected from the group consisting of unsubstituted Chalcones, mon-substituted chalcones, di-substituted chalcones, tri-substituted chalcones, and mixture thereof; flavones selected from the group consisting of unsubstituted flavones, mono-substituted flavones, di-substituted flavones, and mixtures thereof; oe or more isoflavones; coumarins selected from the group consisting of unsubstituted coumarins, mono-substituted coumarins, di-substituted coumarins, and mixtures thereof; flavonols, anthocyanins, catechins, proanthocyanidins (Grape seed extract). Flavonoids which are broadly disclosed in U.S. Pat. Nos. 5,686,082 and 5,686,367 can also be used;
chlorogenic acid and ferulic acid.

The present compositions, e.g. those containing an antioxidant of formulae (1), (2) and/or (3), and optionally a further active ingredient such as a vitamin, are useful for the treatment of inflammatory and allergic conditions (e.g. as described by Skaper et al., Free Radical Biology and Medicine 22, 669-78 (1997)), as well as for the treatment of conditions involving disturbances of cell proliferation. They are preferably used for the local treatment of radical-induced adverse reactions such as inflammatory and allergic conditions collagen damage, reperfusion damage, DNA-damage; especially for the skin treatment.

In vitro assays show that the present compositions inhibit the formation of different mediators that are an important factor in inflammation.

The hindered nitroxyl, hydroxylamine and hydroxylamine salt compounds (3) are for example of formulae A to EE and A* to EE* as described in IPCOM000130489D.

C₁-C₂₂Alkyl is straight-chain or branched alkyl radicals, such as methyl, ethyl, n-propyl, iso-propyl, n-butyl, sec-butyl, tert-butyl, amyl, isoamyl or tert-amyl, heptyl, octyl, isooctyl, nonyl, decyl, undecyl, dodecyl, tetradecyl, pentadecyl, hexadecyl, heptadecyl, octadecyl or eicosyl.

C₁-C₂₂Alkylthio is straight-chain or branched alkylthio radicals, such as methylthio, ethylthio, n-propylthio, isopropylthio, n-butylthio, sec-butylthio, tert-butylthio, amylthio, heptylthio, octylthio, isooctylthio, nonylthio, decylthio, undecylthio, dodecylthio, tetradecylthio, pentadecylthio, hexadecylthio, heptadecylthio, octadecylthio or eicosylthio.

C₂-C₂₂Alkylthioalkyl is alkylthio as described above attached by its sulfur atom to alkyl, where the total residue contains 2-22 carbon atoms.

C₂-C₁₈Alkenyl is, for example, allyl, methallyl, isopropenyl, 2-butenyl, 3-butenyl, isobutenyl, n-penta-2,4-dienyl, 3-methyl-but-2-enyl, n-oct-2-enyl, n-dodec-2-enyl, isododecenyl, n-dodec-2-enyl or n-octadec-4-enyl.

C₅-C₇Cycloalkyl is cyclopentyl, cycloheptyl or, preferably, cyclohexyl.

C₇-C₉-Phenylalkyl includes phenylpropyl (such as cumyl), phenylethyl and, preferably, benzyl.

M as alkali often is Li, Na, K, Cs.

Useful compounds of present formulae (1) and (2) include those listed in Table 1:

TABLE 1

| compound of | |
|---|---|
| formula | |
| (7) | 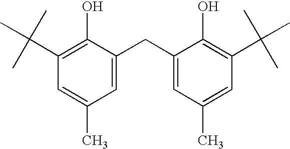 |
| (8) | 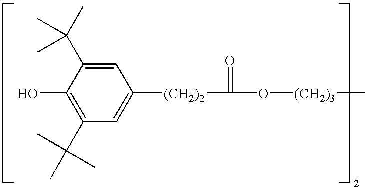 |
| (9) | 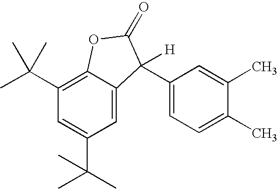 |
| (10) | 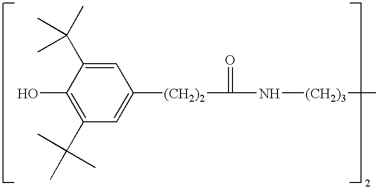 |
| (11) | 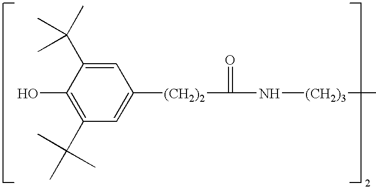 |
| (12) | 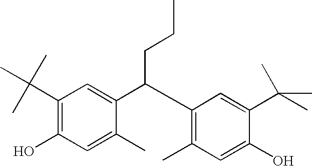 |
| (13) | 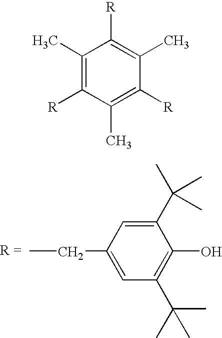 |
| (14) | 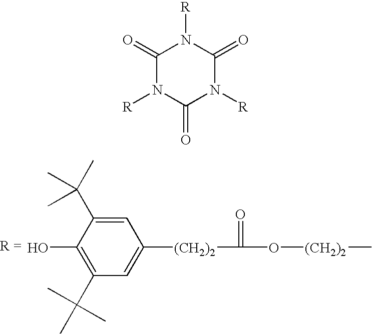 |
| (15) | 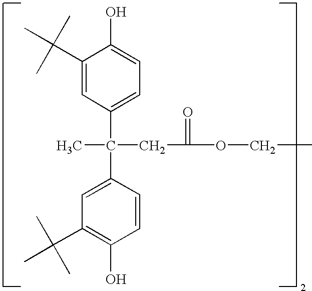 |
| (16) | 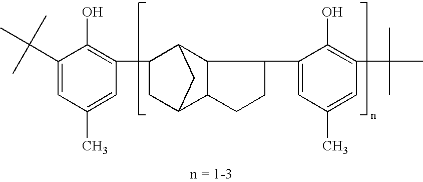 |
| (17) | 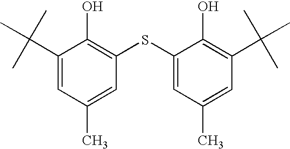 |
| (18) | 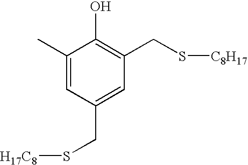 |
| (19) | 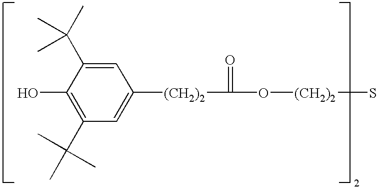 |
| (20) | 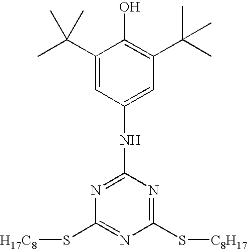 |
| (21) | 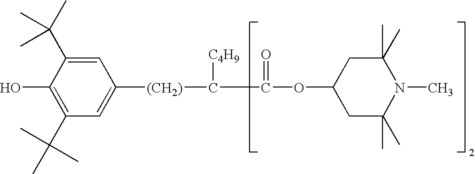 |
| (22) | 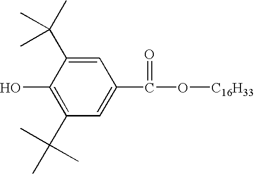 |
| (23) | 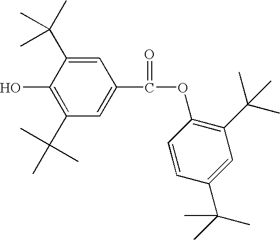 |
| (24) | 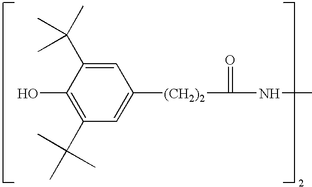 |
| (25) | 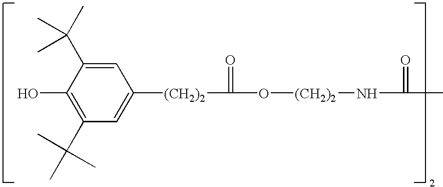 |
| (26) | 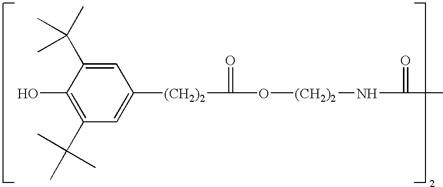 |
| (27) | 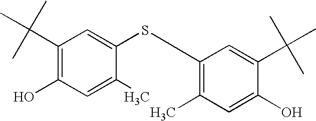 |
| (28) | 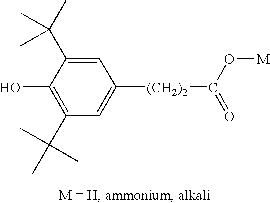 |
| (29) | 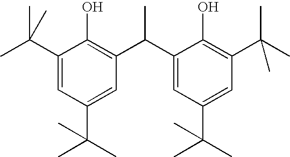 |
| (30) | 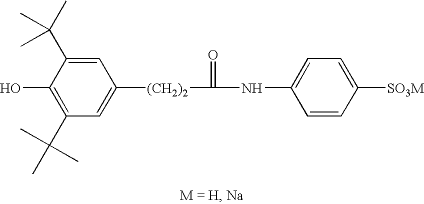 |
| (31) | 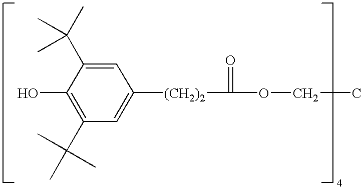 |
| (32) | 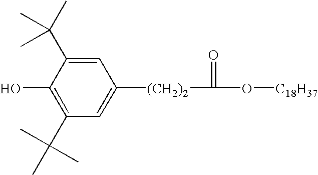 |
| (33) | 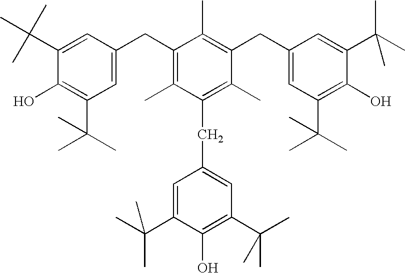 |

Most preferred antioxidants are compounds Nos. 30 (referred to as AO6 in the below examples) and 31 (referred to as AO7 in the below examples).

Useful hindered amine compounds (3) include those mentioned in IPCOM000130489D; preferred hindered nitroxyl, hydroxylamine and hydroxylamine salt compounds of formula (3) are selected from 1-oxyl-2,2,6,6-tetramethyl-4-hydroxypiperidine; 1-hydroxy-2,2,6,6-tetramethyl-4-hydroxypiperidine; 1-hydroxy-2,2,6,6-tetramethyl-4-hydroxypiperidinium chloride; 1-hydroxy-2,2,6,6-tetramethyl-4-hydroxypiperidinium acetate; 1-hydroxy-2,2,6,6-tetramethyl-4-hydroxypiperidinium bisulfate; 1-hydroxy-2,2,6,6-tetramethyl-4-hydroxypiperidinium citrate; bis(1-hydroxy-2,2,6,6-tetramethyl-4-hydroxypiperidinium) citrate; tris(1-hydroxy-2,2,6,6-tetramethyl-4-hydroxypiperidinium) citrate; tetra(1-hydroxy-2,2,6,6-tetramethyl-4-hydroxypiperidinium) ethylenediaminetetraacetate; tetra(1-hydroxy-2,2,6,6-tetramethyl-4-acetamidopiperidinium) ethylenediaminetetraacetate; tetra(1-hydroxy-2,2,6,6-tetramethyl-4-oxopiperidinium) ethylenediaminetetraacetate; penta(1-hydroxy-2,2,6,6-tetramethyl-4-hydroxypiperidinium) diethylenetriaminepentaacetate; penta(1-hydroxy-2,2,6,6-tetramethyl-4-acetamidopiperidinium) diethylenetriaminepentaacetate; and penta(1-hydroxy-2,2,6,6-tetramethyl-4-oxopiperidinium) diethylenetriaminepentaacetate. For example, the compounds of formula (3) are hydroxylamine salts selected from 1-hydroxy-2,2,6,6-tetramethyl-4-hydroxypiperidinium citrate; bis(1-hydroxy-2,2,6,6-tetramethyl-4-hydroxypiperidinium) citrate; tris(1-hydroxy-2,2,6,6-tetramethyl-4-hydroxypiperidinium) citrate; 1-hydroxy-2,2,6,6-tetramethyl-4-hydroxypiperidinium DTPA; bis(1-hydroxy-2,2,6,6-tetramethyl-4-hydroxypiperidinium) DTPA; tris(1-hydroxy-2,2,6,6-tetramethyl-4-hydroxypiperidinium) DTPA; tetrakis(1-hydroxy-2,2,6,6-tetramethyl-4-hydroxypiperidinium) DTPA; pentakis(1-hydroxy-2,2,6,6-tetramethyl-4-hydroxypiperidinium) DTPA; 1-hydroxy-2,2,6,6-tetramethyl-4-hydroxypiperidinium EDTA; bis(1-hydroxy-2,2,6,6-tetramethyl-4-hydroxypiperidinium) EDTA; tris(1-hydroxy-2,2,6,6-tetramethyl-4-hydroxypiperidinium) EDTA; tetrakis(1-hydroxy-2,2,6,6-tetramethyl-4-hydroxypiperidinium) EDTA; 1-hydroxy-2,2,6,6-tetramethyl-4-oxo-piperidinium citrate; bis(1-hydroxy-2,2,6,6-tetramethyl-4-oxo-piperidinium) citrate; tris(1-hydroxy-2,2,6,6-tetramethyl-4-oxo-piperidinium) citrate; 1-hydroxy-2,2,6,6-tetramethyl-4-oxo-piperidinium DTPA; bis(1-hydroxy-2,2,6,6-tetramethyl-4-oxo-piperidinium) DTPA; tris(1-hydroxy-2,2,6,6-tetramethyl-4-oxo-piperidinium) DTPA; tetrakis(1-hydroxy-2,2,6,6-tetramethyl-4-oxo-piperidinium) DTPA; pentakis(1-hydroxy-2,2,6,6-tetramethyl-4-oxo-piperidinium) DTPA; 1-hydroxy-2,2,6,6-tetramethyl-4-oxo-piperidinium EDTA; bis(1-hydroxy-2,2,6,6-tetramethyl-4-oxo-piperidinium) EDTA; tris(1-hydroxy-2,2,6,6-tetramethyl-4-oxo-piperidinium) EDTA; tetrakis(1-hydroxy-2,2,6,6-tetramethyl-4-oxo-piperidinium) EDTA; 1-hydroxy-2,2,6,6-tetramethyl-4-acetamidopiperidinium citrate; bis(1-hydroxy-2,2,6,6-tetramethyl-4-acetamidopiperidinium) citrate; tris(1-hydroxy-2,2,6,6-tetramethyl-4-acetamidopiperidinium) citrate; 1-hydroxy-2,2,6,6-tetramethyl-4-acetamidopiperidinium DTPA; bis(1-hydroxy-2,2,6,6-tetramethyl-4-acetamidopiperidinium) DTPA; tris(1-hydroxy-2,2,6,6-tetramethyl-4-acetamidopiperidinium) DTPA; tetrakis(1-hydroxy-2,2,6,6-tetramethyl-4-acetamidopiperidinium) DTPA; pentakis(1-hydroxy-2,2,6,6-tetramethyl-4-acetamidopiperidinium) DTPA; 1-hydroxy-2,2,6,6-tetramethyl-4-acetamidopiperidinium EDTA; bis(1-hydroxy-2,2,6,6-tetramethyl-4-acetamidopiperidinium) EDTA; tris(1-hydroxy-2,2,6,6-tetramethyl-4-acetamidopiperidinium) EDTA; tetrakis(1-hydroxy-2,2,6,6-tetramethyl-4-hydroxypiperidinium) EDTA; 1-hydroxy-2,2,6,6-tetramethyl-4-acetoxypiperidinium citrate; bis(1-hydroxy-2,2,6,6-tetramethyl-4-acetoxypiperidinium) citrate; tris(1-hydroxy-2,2,6,6-tetramethyl-4-acetoxypiperidinium) citrate; 1-hydroxy-2,2,6,6-tetramethyl-4-acetoxypiperidinium DTPA; bis(1-hydroxy-2,2,6,6-tetramethyl-4-acetoxypiperidinium) DTPA; tris(1-hydroxy-2,2,6,6-tetramethyl-4-acetoxypiperidinium) DTPA; tetrakis(1-hydroxy-2,2,6,6-tetramethyl-4-acetoxypiperidinium) DTPA; pentakis(1-hydroxy-2,2,6,6-tetramethyl-4-acetoxypiperidinium) DTPA; 1-hydroxy-2,2,6,6-tetramethyl-4-acetoxypiperidinium EDTA; bis(1-hydroxy-2,2,6,6-tetramethyl-4-acetoxypiperidinium) EDTA; tris(1-hydroxy-2,2,6,6-tetramethyl-4-acetoxypiperidinium) EDTA and tetrakis(1-hydroxy-2,2,6,6-tetramethyl-4-acetoxypiperidinium) EDTA.

The above named counter-ions are ethylenediaminetetraacetic acid (EDTA), diethylenetriaminepentaacetic acid (DTPA), hydroxyethylethylenediaminetriacetic acid (HEDTA), nitrilotriacetic acid (NTA) or diethylenetriaminepentamethylenephosphonic acid (DTPMPA).

G₁ and G₂ preferably are, independently of each other, C₁-C₁₋₈alkyl and, in particular, C₁-C₅alkyl, especially tert.-butyl. In preferred compounds of the formula (1) or (2), G₁ is located in meta-position relative to G₂.

G₃ is most preferred as hydrogen; C₁-C₂₂alkyl; SO₃M; propyl substituted by OH and/or by C₂-C₂₂alkanoyloxy; a direct bond; —CH₂—;

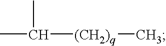

propyl substituted by OH or C₂-C₂₂alkanoyloxy;

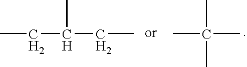

Preferred G₄ and G₅ independently are H or C₁-C₄alkyl, especially methyl.

Of specific technical interest are those compounds of the formula (1) containing a thio group, i.e. those wherein G₁ and/or G₃ are selected from alkylthio, alkylthioalkyl, —S—.

It is preferred to use compounds of formula (1) and/or (2), especially wherein
a is 1;
each of b, c, d, e, n, M, q, V is as defined above;
Q, where present, is —CₘH₂ₘ— and, preferably, a methylene or ethylene radical,
T, where present, is —CₙH₂ₙ— or phenylene;
G₃ is hydrogen; C₁-C₂₂alkyl; SO₃M; propyl substituted by OH and/or by C₂-C₂₂alkanoyloxy;
or G₃ is a direct bond; —CH₂—;

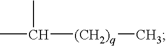

or propyl substituted by OH or C₂-C₂₂alkanoyloxy;
or G₃ is

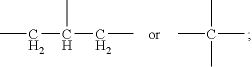

G₄ and G₅ independently are H or C₁-C₄alkyl, especially methyl;
most preferred among them are those compounds of the formula (1), wherein
and G₃ is SO₃M or propyl substituted by OH and/or by C₁₂-C₂₂alkanoyloxy; or G₃ is a direct bond; —CH₂—;

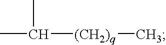

or propylene substituted by OH or C₁₂-C₂₂alkanoyloxy;
or G₃ is

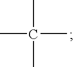

where M is alkali and q is 0 or 1;
Q, where present, is ethylene,
T, where present, is p-phenylene;
or
wherein e is 1 or 2 and G₃ is SO₃M; propyl substituted by OH and/or by C₂-C₂₂alkanoyloxy;
or G₃ is a direct bond; —CH₂—;

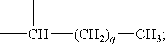

or propyl substituted by OH or C₂-C₂₂alkanoyloxy; where M is alkali and q is 0 or 1;
or of the formula (2).

Most preferred compound of the formula (3) is of the formula

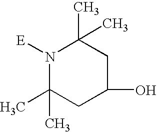

or is an acid addition salt thereof, especially as defined above.

Particularly interesting compounds (1) include those of formula

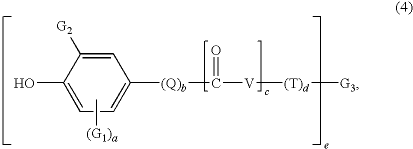

wherein
G₁ and G₂ are each independently of the other C₁-C₅alkyl, or G₁ and G₂ especially are 2,6-ditert.butyl;
a is 1 or 2, especially 1; and
G₃, Q, V, T, b, c, d and e have the meanings cited for formula (1), or especially
b is 1 and Q is ethylene;

### c is 1 and V is O or NH;

d is 1 and T is CH₂ or phenylene; and
e is 1 and G₃ is H. C₁-C₁₋₈alkyl such as methyl or C₁₋₇alkyl, or is SO₃Na; or
e is 4 and G₃ is a carbon atom;
or each of b, c and d is 0, e is 2 and G₃ is methylene or ethylidene.

Most preferred compounds are those of formula (1), especially wherein G₁ and G₂ are the tert-butyl radical; and a is 1.

It is also preferred to use compounds of formula

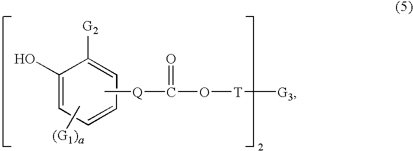

wherein
G₁ and G₂ are each independently of the other C₁-C₅alkyl;

### Q is —CₘH₂ₘ—; or —CₘH₂ₘ—NH—;

G₃ is a direct bond; —O—; —S—; —CH₂—; or

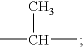

a is 1 or 2;
m is 1 to 5; and
T has the meaning cited in formula (1).

Interesting compounds of formula (1) are those, wherein

Q is ethylene; or

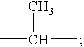

G₃ is a direct bond.

Likewise preferred are compounds of formula

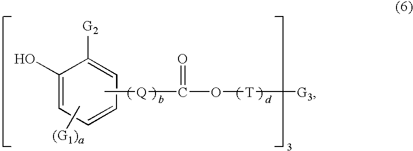

wherein

### Q is —CₘH₂ₘ—;

### T is —CₙH₂ₙ—;

G₁ and G₂ are each independently of the other C₁-C₅alkyl;
G₃ is the radical of formula (1g); (1h); (1i); or (1k);
m and n are each independently of the other 1 to 3;
a is 1 or 2; and
b and d are each independently of the other 0 or 1.

Other antioxidants which are preferably used conform to formula

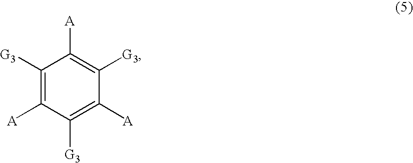

wherein
A is a radical of formula

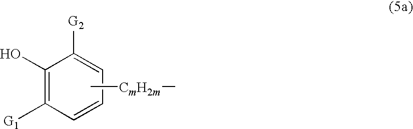

G₁, G₂ and G₃ are each independently of one another C₁-C₅alkyl; and
m is 1 to 3.

Other preferred antioxidants are those of formula

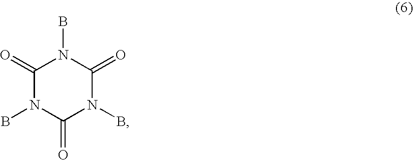

wherein
B is a radical of formula

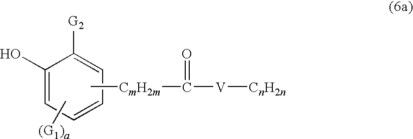

G₁ and G₂ are each independently of the other C₁-C₅alkyl;
V is —O—; or —NH—;
a is 1; or 2;
m is 1 to 3; and
n is 0 to 3.

A number of present antioxidant compounds, e.g. those of formulae (1), (2) and (3), previously have been known as antioxidants for plastics and certain other organic materials.

Within the present invention, they can be used as individual compounds or as mixtures of several individual compounds. Some of the present antioxidants, essentially those of formula (1) wherein e is 2, 3 or especially 4 and those of formula (2), combine high antioxidant activity and tissue/skin compatibility with rather low solubility in water. Encapsulation of these compounds, especially in nanoscaled particles such as described further below, brings about the further advantage that a higher loading and activity of these compounds in the final formulation may be achieved than by mere addition (without encapsulation).

A preferred subject of the invention therefore is a cosmetical or pharmaceutical composition containing an antioxidant of the formula (1) wherein e is 2, 3 or especially 4, or of the formula (2), encapsulated in a lipid particle. Representative examples for these “lipophilic” antioxidants are compounds Nos. 7-15, 17, 19, 23-27, 29, 31 and 33 listed above; most preferred among them are compounds Nos. 9, 29 and especially 31 (denoted in the below examples as AO4, AO4, AO5, AO7).

One of the forms of such a composition is a liquid microemulsion or microdispersion containing the antioxidant loaded particles as the dispersed phase in an aqueous continuous phase. The composition may, however, be in a solid or semisolid form as well, as explained further below. Preferred methods of encapsulation and preparation of such a composition, as well as properties thereof, are also described further below.

Present antioxidants, e.g. those of formulae (1), (2), (3), can also be incorporated together with further components, especially coencapsulated oxidizable and/or lipophilic components such as those described in IPCOM000130489D. Also of importance is the concomitant use of a more “hydrophilic” antioxidant of formula (1), where e is 1 (such as the compound AO6), or of formula (3) and/or a further active ingredient, in free form, e.g. in the continuous phase of a corresponding microemulsion or microdispersion.

The antioxidant component, the optional further active ingredient component, and an optional further component apart from the carrier, each is usually contained within the concentration range from 0.0001% to 10% of the total weight of the preparation/composition %, and most preferably from 0.0005 to 5% by weight of the total composition. If an active ingredient is present, the weight ratio of the antioxidant:active ingredient in the preparation/composition usually ranges from 1:10 to 10:1, preferably is larger than 1:1 such as from the range 1:1 to 10:1.

Of special technical interest is a preparation containing a vitamin such as vitamin C in free or modified form in an amount of at least 0.05% by weight, such as 0.05-1.0% by weight, and 0.05 to 2.0% by weight, preferably the same amount or more, of an antioxidant, with the encapsulated antioxidant being present in an amount of at least 0.05% by weight of the total preparation.

Further kinds of antioxidants useful for encapsulation according to the present invention include components that interrupt the photochemical reaction chain triggered when UV radiation penetrates the skin or hair. Typical examples of such antioxidants are amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotinoids, carotenes, lycopene and derivatives thereof, chlorogenic acid and derivatives thereof, lipoic acid and derivatives thereof (e.g. dihydrolipoic acid), aurothioglycose, propylthiouracil and other thiols (e.g. thioredoxin, glutathione, cysteine, cystine, cystamine and the glycosyl, N-acetyl, methyl, ethyl, propyl, amyl, butyl, lauryl, palmitoyl, oleyl, linoleyl, cholesteryl and glyceryl esters thereof) and also salts thereof, dilauryl thiodipropionate, distearyl thiodipropionate, thiodipropionic acid and derivatives thereof (esters, ethers, peptides, lipids, nucleotides, nucleosides and salts) and also sulfoximine compounds (e.g. buthionine sulfoximines, homocysteine sulfoximine, buthionine sulfones, penta-, hexa-, hepta-thionine sulfoximine), also (metal) chelating agents (e.g. hydroxy fatty acids, palmitic acid phytic acid, lactoferrin, chitosan and derivatives such as phosphonomethylated chitosan) and preferably those disclosed in U.S. Pat. No. 5,487,884; WO91/16035; WO91/16034; hydroxy acids (e.g. citric acid, lactic acid, malic acid), humic acid, bile acid, bile extracts, bilirubin, biliverdin, EDTA, EDDS, EGTA and derivatives thereof, unsaturated fatty acids and derivatives thereof (e.g. linolenic acid, linoleic acid, oleic acid), folic acid and derivatives thereof, ubiquinone and ubiquinol and derivatives thereof, vitamin C and derivatives (e.g. ascorbyl palmitate, magnesium ascorbyl phosphate, ascorbyl acetate), tocopherols and derivatives (e.g. vitamin E acetate), vitamin A and derivatives (e.g. vitamin A palmitate) and also coniferyl benzoate of benzoin resin, rutinic acid and derivatives thereof, glycosylrutin, ferulic acid, furfurylidene glucitol, carnosine, butyl hydroxytoluene, butyl hydroxyanisole, nordihydroguaiaretic acid, trihydroxybutyrophenone, uric acid and derivatives thereof, mannose and derivatives thereof, superoxide dismutase, N-[3-(3,5-di-tert-butyl-4-hydroxyphenyl)propionyl]sulfanilic acid (and salts thereof, for example the disodium salts), zinc and derivatives thereof (e.g. ZnO, ZnSO₄), selenium and derivatives thereof (e.g. selenium methionine), stilbene and derivatives thereof (e.g. stilbene oxide, trans-stilbene oxide) and the derivatives suitable according to the invention (salts, esters, ethers, sugars, nucleotides, nucleosides, peptides and lipids) of those mentioned active ingredients. Further HALS (“Hindered Amine Light Stabilizers”) compounds may also be mentioned.

Further synthetic and natural antioxidants are listed e.g. in WO 00/25731 (see e.g. structures 1-3 (page 2), structure 4 (page 6), structures 5-6 (page 7) and compounds 7-33 (page 8-14)).

The preparation for topical application may additionally contain at least one further component with anti-inflammatory effect as active ingredient, preferably from 0.01% to 10% more preferably about 0.5% to about 5%, of the composition, such as:
steroidal anti-inflammatory agents, including but not limited to, corticosteroids such as hydrocortisone and their derivatives;
non-steroidal anti-inflammatory agents, including but not limited to, oxicams, salycilates, acetic acid derivatives, fenamates, propionic acid derivatives, pyrazoles;
natural anti-inflammatory agents including, but not limited to, α-bisabolol, allantoin, lyophilized extract of aloe vera, panthenol, betulin, compounds of the Licorice (Glycyrrhiza glabra) including glycyrrhetic acid, glycyrrhizic acid, and derivatives thereof (salts and esters) suc as sodium glycyrrhizinate, potassium glycyrrhizinate, ammonium glycyrrhizinate; botulinic acid, alkaline salts thereof and salts of alkaline-earth metals, boswellic acid, alkaline salts thereof and salts of alkaline-earth metals, rosemaric acid, alkaline salts thereof and salts of alkaline-earth metals; polynonsatured fatty acids, as linoleic (18:2n6), α-linolenic (18:3n3), γ-linolenic (18:3n6), octadekanetetraenic (18:4n3), dihomo-γ-linolenic (20:3n6), eikosantetraenic (20:4n3), arachidonic (20:4n6), eikosanpentaenic (20:5n3) acids and esters thereof with alcohols of the general formula

R1(CH2)m-(CHOH)—(CH2)nR2 (5)

where R1 and R2 are hydrogen atoms or OH group, m, n are integral numbers from 0 to 17 where m+n sum is maximally 21;
phytosterols and their polyethoxylate derivatives of the general formulas (6) and (7) below, where R is isoalkyl or isoalkenyl group with 8-10 carbon atoms, where n is integral number from 0 to 50, especially campesterol, β-sitosterol, stigmasterol, cholesterol, Δ-5-avenasterol, Δ-7-avenasterol, brassicasterol, spinasterol and fukosterol

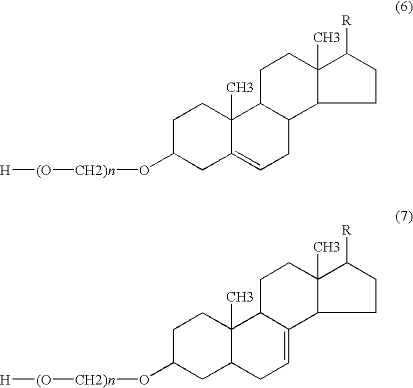

The cosmetical or pharmaceutical compositions include liquid, semisolid or solid preparations.

Examples of liquid compositions are injectable solutions, infusion solutions, drops, sprays, aerosols, emulsions, lotions, suspensions, drinking solutions, drinking gargles and in-halants.

Examples of semisolid compositions are ointments, creams (O/W emul-sions), rich creams (W/O emulsions), gels, lotions, foams, pastes, suspensions, ovula, plasters, including applications for transdermal systems such as masks, compresses, pads.

Examples of solid compositions are tablets, coated tablets, capsules, gra-nules, effervescent granules, effervescent tablets, lozenges, sucking and chewing tablets, suppositories, implants, lyophilisates, adsorbates or powders.

The present compositions may also be used as food additives or dietary food or supplements, functional food, enriched foods, or for the preparation thereof.

Preferred are liquid or semisolid preparations.

Galenic compositions comprising the present compounds will be understood as meaning in particular emulsions, ointments, gels, sprays and powders; for example creams, gels, lotions, alcoholic and aqueous/alcoholic solutions, emulsions, wax/fat compositions, stick preparations such as lip sticks or deodorants, powders or ointments.

The final formulations listed may exist in a wide variety of presentation forms, for example:
in the form of liquid preparations as a W/O, O/W, O/W/O, W/O/W or PIT emulsion and all kinds of microemulsions,
in the form of a gel,
in the form of an oil, a cream, milk or lotion,
in the form of a powder, a lacquer, a tablet or make-up,
in the form of a stick,
in the form of a spray (spray with propellent gas or pump-action spray) or an aerosol,
in the form of a foam, or
in the form of a paste.

Several particulate formulations, especially skin care delivery systems, are:

A] Nanoemulsions and nanoparticles are mainly based on lecithin or fractionated phospholipids, especially
nanoemulsions with particle size range of 20 nm-50 nm represented by a single layer membrane, including Nanotopes (lipid core surrounded by a membrane composed of phospholipids and co-surfactants; stable and small size particle (smaller than liposomes) due to the intercalation of co-surfactant between the extending lecithin molecules) and Nanosomes;
nanoparticles with particle size range of 100 nm-300 nm represented by a bi-layer membrane; such as Liposomes, i.e. spherical vesicles comprising amphiphilic lipids (predominantly phospholipids) enclosing an aqueous core and forming one or several concentric bi-layers; small unilamellar vesicles (SUV), large unilamellar vesicles (LUV), large multilamellar vesicles (MLV) or multivesicullar vesicles (MVV)).

A nanoemulsion or nanodispersion creates a large surface-to-volume ratio for emulsion particles that contact skin. Thus, more active ingredients contact the skin at the surface-to-surface interaction between the emulsion and the skin. The components of the invention may, for example, be incorporated into the bi-layers, into the aqueous core or distributed in both.

B] Microcapsules with particle size>1 μm based on matrix or encapsulation layer (spherical system based on a core material containing the active; the core is, then, surrounded by one or several coating layers or shells).

Polymers used to form those microcapsules include natural gums, cellulosic ingredients, polysaccharides, synthetic polyacrylates or polyacrylamides, polyvinyl alcohol (PVA), lipids, inorganics (silicates/clays), high molecular weight proteins such as gelatin, albumin etc.; examples include Nylon micro-porous spheres (Orgasol range from Elf Atochem), Mineral fillers such as sericite surface-treated by bifunctional coating (reaction between reactive fatty acid derivatives and the aqueous solution of sericite); Glycospheres (core based on modified starch and outer lipid membrane based on fatty acids and polar lipids); carbon nanotubes; Silica shells, made of silicates, for non aqueous and solid end-products such as sticks, dry powders etc.

C] Matrix particulate systems which entrap the active ingredient within the uniform core matrix; examples:
Solid Lipid Nanoparticle (SLN) technology based only on solid lipids;
Nanostructured Lipid Carriers (NLC) made of blend of solid lipid and liquid lipid (typical particle size diameter within the range 80 nm-1 μm);
Nanospheres (e.g. U.S. Pat. No. 6,491,902) represented by solid hydrophobic and highly cationic nanospheres (typical particle size range 10 nm to 1 μm) and based on solid hydrophobic matrix coated with highly cationic or bioadhesive layer.
   D] Multi-walled delivery systems, e.g. systems similar to liposome structures but made only of non-phospholipidic “membrane-mimetic” amphiphiles such as oleic acid, saturated or unsaturated fatty acids, long-chain soaps combined with non ionic surfactants, derivatives of polyglycerol, di-ammonium amphiphiles, cationic surfactants, cationic amphiphiles involving aminoacid residues, sucrose fatty acid esters, aqueous mixture of anionic and cationic surfactants.
   E] Microsponge technology such as a system based on microscopic polymer-based sphere that consist of interconnecting voids within a non collapsible structure (non continuous shell); examples include a copolymer of styrene and divinylbenzene, or vinyl derivatives, water-swellable particles made of lactose, cellulose and cellulose derivatives such as Unispheres from Induchem etc.
   F] Silicone-based vesicles such as multi-layers vesicles similar to liposome structures, where layers are made of polyether-modified dimethicone (dimethicone copolyol), silicone elastomers, blends of dimethicone crosspolymer, dimethicone/vinyidimethicone crosspolymer or PEG-modified dimethicone crosspolymer etc.
   G] Cyclodextrins, usually oligomeric and cyclic carbohydrate compounds containing 6 to 8 glucose units such as α-, β- and γ-cyclodextrin.

According to a preferred method, the present compounds are applied in nanodispersed or encapsulated form of type [A] mentioned above, such as described in US-2005-0191330 (see especially sections [0007-0079], [0120-0133] and examples) and US-2003-0190347 (see especially sections [0005-0087] and examples).

The invention therefore includes a method for the encapsulation of an antioxidant, especially for the protection of an active ingredient, as initially described, wherein the encapsulation is achieved by mixing

(a) a membrane-forming molecule,
(b) a coemulsifier and
(c) a lipophilic component comprising an antioxidant,
until a homogeneous liquid is obtained.

The liquid thus obtained is then added to a water phase.

Component (a) is advantageously selected from the phospholipid classes or corresponding components described in US-2005-0191330 or US-2003-0190347.

Component (b) is preferably selected from polyethoxylated sorbitan fatty acid esters, polyethoxylated fatty alcohols, polyethoxylated fatty acids, polyethoxylated vitamin E derivatives, polyethoxylated lanoline or lanoline derivatives, polyethoxylated fatty acid glycerides and partial glycerides, polyethoxylated alkylphenols, sulfuric acid semiesters of a polyethoxylated fatty alcohol or salt thereof, polyethoxylated fatty amines or amides, polyethoxylated carbon hydrates; the antioxidant of component (c) is preferably selected from compounds AO3-AO8 described further below, and especially are preferred as AO6 or AO7.

Lipophilic component (c) may also comprise the active ingredient, if this is to be coencapsulated together with the antioxidant for best protection. The lipophilic component often further comprises a cosmetically acceptable lipid; this may be selected, for example, from mono-, di- or especially triglycerides, especially of fatty esters, and/or other components such as (INCI-names): Propylene glycol, C₁₂₋₁₅ alkyl benzoate, Diisopropyl adipate, Dioctyl adipate, Di-2-ethylhexyl succinate, Propylene glycol dicaprylate, Octyl octanoate Dioctyl terephthalate, Trioctyl citrate, Trioctanoin⁺, Octyl dodecanol, Caprylyl pyrrolidone, Triethyl citrate, 2-Butyl-1-octanol, Propylene carbonate, Dibutyl carbonate, Neopentyl glycol dioctanoate. Preferred for the antioxidant of formula 31 (i.e. AO7), for example, is a triglyceride (e.g. a C₄-C₁₈fatty acid triglyceride like those of the Miglyol®-series; CONDEA), or Diisopropyl adipate, Dioctyl adipate, Di-2-ethylhexyl succinate, Caprylyl pyrrolidone, Dibutyl carbonate.

The weight ratio of the antioxidant:cosmetically acceptable lipid in the prephase usually ranges from 1:10 to 10:1, preferably from 1:5 to 5:1.

The homogenous liquid thus obtained (i.e. the “prephase”) usually is clear (unless clouded by the active ingredient). It may contain one or more further components (d) such as an alcohol. In a preferred method, the homogeneous liquid obtained after mixing components (a), (b), (c) and optionally (d) is essentially anhydrous.

In a preferred method,

(a) 5 to 20% by weight of a phospholipid,
(b) 15 to 40% by weight of a coemulsifier,
(c) 30 to 70% by weight of a lipophilic component consisting of an antioxidant of formulae (1) to (3) as described above, and optionally a cosmetically acceptable lipid and/or an active ingredient selected from oxidizable natural substances, vitamins, plant extracts, fragrances; and optionally
(d) 0 to 30%, especially 3 to 30%, by weight of an alcohol, such as a C₁-C₅alkanol or C₁-C₅alkanediol, especially ethanol,
are mixed, with the sum of percentages of the components (a), (b), (c) and optionally (d) adding to 100%.

As described in US-2005-0191330 and US-2003-0190347 mentioned above, a microemulsion or microdispersion prepared in this way usually is obtained, for practical reasons, first as a concentrate, which is then employed for the preparation of the final ready-for-use preparation. The preparation step from the prephase to the concentrate usually brings about a dilution of the prephase of 1:5 to about 1:20. The concentrate may be further diluted (i.e. combined with further components) to obtain the final product; this is often done in the same ratio of 1:5 to about 1:20. Thus, the final product contains the ingredients of the prephase usually in a dilution from about 1:25 to about 1:400 or, more often, in a dilution of about 1:50 to about 1:200, free components added as part of a diluting phase not included. Formulations as described in the present invention encompass prephase, concentrate as well as the final formulation ready for end use.

The nanodispersions obtained in the above process contain particles usually having an average diameter of less than 80 nm (e.g. 10-80 nm), typically of less than 50 nm, and preferably about 30 nm or less. The distribution typically is monodisperse and corresponds to a Gaussian distribution.

An example for a cosmetical or pharmaceutical composition is a nanodispersion, which is obtained by

mixing of
(a) 5 to 20% by weight of a phospholipid,
(b) 15 to 40% by weight of a coemulsifier,
(c) 30 to 70% by weight of a lipophilic component consisting of a triglyceride, an antioxidant of formulae (1) to (3), especially of formula (AO3), (AO4), (AO5), (AO6), (AO7), (AO8) as described below, and an active ingredient selected from oxidizable natural substances, vitamins, plant extracts, fragrances; and
(d) 3 to 30% by weight of an alcohol,
with the sum of percentages of the components (a), (b), (c) and (d) adding to 100%, followed by addition of the liquid thus obtained to a water phase. The composition thus obtained usually is then supplemented by further components, e.g. those important for the specific end use.

The composition may contain further components, e.g. as listed in WO00/25731, WO03/103622, IPCOM000130489D, or as mentioned in the below formulations.

Examples for end formulations containing the composition of the invention also include
skin-care preparations, e.g. skin-washing and cleansing preparations in the form of tablet-form or liquid soaps, soapless detergents or washing pastes,
bath preparations, e.g. liquid (foam baths, milks, shower preparations) or solid bath preparations, e.g. bath cubes and bath salts;
skin-care preparations, e.g. skin emulsions, multi-emulsions or skin oils;
personal care preparations, e.g. facial make-up in the form of day creams or powder creams, face powder (loose or pressed), rouge or cream make-up, eye-care preparations, e.g. eyeshadow preparations, mascara, eyeliner, eye creams or eye-fix creams; lip-care preparations, e.g. lipsticks, lip gloss, lip contour pencils, nail-care preparations, such as nail varnish, nail varnish removers, nail hardeners or cuticle removers;
foot-care preparations, e.g. foot baths, foot powders, foot creams or foot balsams, special deodorants and antiperspirants or callus-removing preparations;
light-protective preparations, such as sun milks, lotions, creams or oils, sunblocks or tropicals, pre-tanning preparations or after-sun preparations;
skin-tanning preparations, e.g. self-tanning creams;
depigmenting preparations, e.g. preparations for bleaching the skin or skin-lightening preparations including rinse off preparations such as soaps and leave on preparations such as creams etc.;
medicated bar soaps and liquid containing antifungals, anti bacterials etc.;
insect-repellents, e.g. insect-repellent oils, lotions, sprays or sticks;
deodorants, such as deodorant sprays, pump-action sprays, deodorant gels, sticks or roll-ons;
antiperspirants, e.g. antiperspirant sticks, creams or roll-ons;
preparations for cleansing and caring for blemished skin, e.g. synthetic detergents (solid or liquid), peeling or scrub preparations or peeling masks;
hair-removal preparations in chemical form (depilation), e.g. hair-removing powders, liquid hair-removing preparations, cream- or paste-form hair-removing preparations, hair-removing preparations in gel form or aerosol foams;
shaving preparations, e.g. shaving soap, foaming shaving creams, non-foaming shaving creams, foams and gels, preshave preparations for dry shaving, aftershaves or aftershave lotions;
fragrance preparations, e.g. fragrances (eau de Cologne, eau de toilette, eau de parfum, parfum de toilette, perfume), perfume oils or perfume creams;
hair-treatment preparations, e.g. hair-washing preparations in the form of shampoos and conditioners, hair-care preparations, e.g. pretreatment preparations, hair tonics, styling creams, styling gels, pomades, hair rinses, treatment packs, intensive hair treatments, hair-structuring preparations, e.g. hair-waving preparations for permanent waves (hot wave, mild wave, cold wave), hair-straightening preparations, liquid hair-setting preparations, hair foams, hairsprays, bleaching preparations, e.g. hydrogen peroxide solutions, lightening shampoos, bleaching creams, bleaching powders, bleaching pastes or oils, temporary, semi-permanent or permanent hair colourants, preparations containing self-oxidising dyes, or natural hair colourants, such as henna or camomile.

Of special importance as preparations for the skin are daily care and/or anti-aging preparations, including light-protective preparations, such as sun milks, lotions, creams, oils, sunblocks or topicals, pretanning preparations or after-sun preparations, also skin-tanning preparations, for example self-tanning creams, skin whitener preparations, skin lightener preparations or combinations of such systems. Of particular interest are anti-aging preparations in combination with UV-protecting systems such as dayly care creams, dayly care lotions, dayly care milk and dayly care preparations in the form of a spray.

The compositions/preparations according to the invention may, where appropriate, also contain one or one more additional compounds as described below, especially in preparations for skin treatment; examples include

Fatty alcohols, Esters of fatty acids, Other adjuvants,
Natural or synthetic triglycerides including glyceryl esters and derivatives,
Pearlescent waxes, Hydrocarbon oils,
Silicones or siloxanes (organosubstituted polysiloxanes) such as dimethicone and related substances,
Fluorinated or perfluorinated oils, Emulsifiers, Adjuvants and additives, Super-fatting agents, Anti-wrinkle actives, Skin lightening agents, Surfactants,
Consistency regulators/thickeners and rheology modifiers, Polymers,
Biogenic active ingredients, Deodorising active ingredients, Anti-dandruff agents,
Hydrotropic agents, Preservatives and Bacteria-inhibiting agents, Perfume oils, Colourants, and/or Other adjuvants, such as the components listed in IPCOM000130489D under these headlines.

Present components usually are used in cosmetical or pharmacological compositions with conventional carriers; further useful components include penetration enhancers, for example urea, dextrane, propylene glycol, oleic acid and the like.

The cosmetical or pharmaceutical composition will usually contain each of the present components antioxidant and further ingredient in amounts of 0.001 to 10% by weight, preferably of 0.01 to 5%, especially 0.01 to 2% by weight, of the final ready-to-use formulation. For the treatment of the conditions listed hereinabove, the pharmaceutical composition of this invention may contain, in addition to the present compounds, further pharmaceutical or cosmetic agents, e.g. having antiphlogistic activity, typically including antiinflammatory agents, vitamins, and/or, where appropriate, antipsoriatic agents, further skin actives, cell proliferation regulators, antiallergic, UV protecting, moisturizing, antiageing, gastroprotective, antiasthmatic agents, DNA-protectants.

The composition of this invention may contain antioxidants and/or light stabilisers apart from present formulae (1)-(3), especially UV absorbers. Suitable components of these classes include those described in EP-A-955355, WO00/25730, WO00/25731, WO03/103622, EP-A-1366763.

Examples are components listed below:

Suitable UV filter substances which can be additionally used with the present compounds

p-aminobenzoic acid derivatives, for example 4-dimethylaminobenzoic acid 2-ethylhexyl
ester;
salicylic acid derivatives, for example salicylic acid 2-ethylhexyl ester;
benzophenone derivatives, for example 2-hydroxy-4-methoxybenzophenone and its
5-sulfonic acid derivative;
dibenzoylmethane derivatives, for example 1-(4-tert-butylphenyl)-3-(4-methoxyphenyl)-
propane-1,3-dione;
diphenylacrylates, for example 2-ethylhexyl 2-cyano-3,3-diphenylacrylate, and 3-(benzo-
furanyl) 2-cyanoacrylate;
3-imidazol-4-ylacrylic acid and esters;
benzofuran derivatives, especially 2-(p-aminophenyl)benzofuran derivatives, described in
EP-A-582 189, US-A-5 338 539, US-A-5 518 713 and EP-A-613 893;
polymeric UV absorbers, for example the benzylidene malonate derivatives described in
EP-A-709 080;
cinnamic acid derivatives, for example the 4-methoxycinnamic acid 2-ethylhexyl ester and
isoamyl ester or cinnamic acid derivatives described in US-A-5 601 811 and WO 97/00851;
camphor derivatives, for example 3-(4′-methyl)benzylidene-bornan-2-one, 3-benzylidene-
bornan-2-one, N-[2(and 4)-2-oxyborn-3-ylidene-methyl)-benzyl]acrylamide polymer, 3-(4′-
trimethylammonium)-benzylidene-bornan-2-one methyl sulfate, 3,3′-(1,4-phenylenedi-
methine)-bis(7,7-dimethyl-2-oxo-bicyclo[2.2.1]heptane-1-methanesulfonic acid) and salts,
3-(4′-sulfo)benzylidene-bornan-2-one and salts; camphorbenzalkonium methosulfate;
hydroxyphenyltriazine compounds, for example 2-(4′-methoxyphenyl)-4,6-bis(2′-hydroxy-4′-
n-octyloxyphenyl)-1,3,5-triazine; 2,4-bis{[4-(3-(2-propyloxy)-2-hydroxy-propyloxy)-2-hy-
droxy]-phenyl}-6-(4-methoxyphenyl)-1,3,5-triazine; 2,4-bis{[4-(2-ethyl-hexyloxy)-
2-hydroxy]-
phenyl}-6-[4-(2-methoxyethyl-carboxyl)-phenylamino]-1,3,5-triazine; 2,4-bis{[4-(tris-
(trimethylsilyloxy-silylpropyloxy)-2-hydroxy]-phenyl}-6-(4-methoxyphenyl)-1,3,5-triazine;
2,4-bis{[4-(2″-methylpropenyloxy)-2-hydroxy]-phenyl}-6-(4-methoxyphenyl)-1,3,5-triazine;
2,4-bis{[4-(1′,1′,1′,3′,5′,5′,5′-heptamethyltrisilyl-2″-
methyl-propyloxy)-2-hydroxy]-phenyl}-6-
(4-methoxyphenyl)-1,3,5-triazine; 2,4-bis{[4-(3-(2-propyloxy)-2-hydroxy-propyloxy)-2-
hydroxy]-phenyl}-6-[4-ethylcarboxy)-phenylamino]-1,3,5-triazine;
benzotriazole compounds, for example 2,2′-methylene-bis(6-(2H-benzotriazol-2-yl)-4-
(1,1,3,3-tetramethylbutyl)-phenol;
trianilino-s-triazine derivatives, for example 2,4,6-trianiline-(p-carbo-2′-ethyl-1′-oxy)-1,3,5-
triazine and the UV absorbers disclosed in US-A-5 332 568, EP-A-517 104, EP-A-507 691,
WO 93/17002 and EP-A-570 838;
2-phenylbenzimidazole-5-sulfonic acid and salts thereof;
menthyl o-aminobenzoates;
physical sunscreens coated or not as titanium dioxide, zinc oxide, iron oxides, mica, MnO,
Fe₂O₃, Ce₂O₃, Al₂O₃, ZrO₂. (surface coatings:
polymethylmethacrylate, methicone
(methylhydrogenpolysiloxane as described in CAS 9004-73-3), dimethicone, isopropyl
titanium triisostearate (as described in CAS 61417-49-0), metal soaps as magnesium
stearate (as described in CAS 4086-70-8), perfluoroalcohol phosphate as C9-15
fluoroalcohol phosphate (as described in CAS 74499-44-8; JP 5-86984, JP 4-330007)).
The primary particle size is an average of 15 nm-35 nm and the particle size in dispersion is
in the range of 100 nm-300 nm.
aminohydroxy-benzophenone derivatives disclosed in DE 10011317, EP 1133980 and EP
1046391
phenyl-benzimidazole derivatives as disclosed in EP 1167358
the UV absorbers described in “Sunscreens”, Eds. N. J. Lowe, N. A. Shaath, Marcel Dekker,
Inc. , New York and Basle or in Cosmetics & Toiletries (107), 50 ff (1992) also can be used
as additional UV protective substances.

| No. | Chemical Name | CAS No. |
|---|---|---|
| 1 | (+/−)-1,7,7-trimethyl-3-[(4-methylphenyl)methylene]bicyclo[2.2.1]- | 36861-47-9 |
| | heptan-2-one; p-methyl benzylidene camphor | |
| 2 | 1,7,7-trimethyl-3-(phenylmethylene)bicyclo[2.2.1]heptan-2-one; | 15087-24-8 |
| | benzylidene camphor | |
| 3 | (2-Hydroxy-4-methoxyphenyl)(4-methylphenyl)methanone | 1641-17-4 |
| 4 | 2,4-dihydroxybenzophenone | 131-56-6 |
| 5 | 2,2′,4,4′-tetrahydroxybenzophenone | 131-55-5 |
| 6 | 2-Hydroxy-4-methoxy benzophenone | 131-57-7 |
| 7 | 2-Hydroxy-4-methoxy benzophenone-5-sulfonic acid | 4065-45-6 |
| 8 | 2,2′-dihydroxy-4,4′-dimethoxybenzophenone | 131-54-4 |
| 9 | 2,2′-Dihydroxy-4-methoxybenzophenone | 131-53-3 |
| 10 | Alpha-(2-oxoborn-3-ylidene)toluene-4-sulphonic acid and its salts; | 56039-58-8 |
| | Mexoryl SL | |
| 11 | 1-[4-(1,1-dimethylethyl)phenyl]-3-(4-methoxyphenyl)propane-1,3- | 70356-09-1 |
| | dione; avobenzone | |
| 12 | Methyl N,N,N-trimethyl-4-[(4,7,7-trimethyl-3-oxobicyclo[2,2,1]hept-2- | 52793-97-2 |
| | ylidene)methyl]anilinium sulphate; Mexoryl SO | |
| 22 | 3,3,5-Trimethyl cyclohexyl-2-hydroxy benzoate; homosalate | 118-56-9 |
| 23 | Isopentyl p-methoxycinnamate; isoamyl methoxy cinnamate | 71617-10-2 |
| 27 | Menthyl-o-aminobenzoate | 134-09-8 |
| 28 | Menthyl salicylate | 89-46-3 |
| 29 | 2-Ethylhexyl 2-cyano,3,3-diphenylacrylate; Octocrylene | 6197-30-4 |
| 30 | 2- ethylhexyl 4- (dimethylamino)benzoate | 21245-02-3 |
| 31 | 2- ethylhexyl 4- methoxycinnamate; octyl methoxy cinnamate | 5466-77-3 |
| 32 | 2-ethylhexyl salicylate | 118-60-5 |
| 33 | Benzoic acid, 4, 4′, 4″- (1, 3, 5- triazine- 2, 4, 6-triyltriimino)tris-, | 88122-99-0 |
| | tris(2-ethyl hexyl)ester; 2,4,6-Trianilino-(p-carbo-2′-ethylhexyl-1′-oxi)- | |
| | 1,3,5-triazine; octyl triazone | |
| 34 | 4- aminobenzoic acid | 150-13-0 |
| 35 | Benzoic acid, 4-amino-, ethyl ester, polymer with oxirane | 113010-52-9 |
| 38 | 2- phenyl- 1H- benzimidazole- 5- sulphonic acid; | 27503-81-7 |
| | phenylbenzimidazolsulfonic acid | |
| 39 | 2-Propenamide, N-[[4-[(4,7,7-trimethyl-3-oxobicyclo[2.2.1]hept-2- | 147897-12-9 |
| | ylidene)methyl]phenyl]methyl]-, homopolymer | |
| 40 | Triethanolamine salicylate | 2174-16-5 |
| 41 | 3, 3′-(1,4-phenylenedimethylene)bis[7, 7-dimethyl- 2-oxo- | 90457-82-2 |
| | bicyclo[2.2.1]heptane-1 methanesulfonic acid]; Cibafast H | |
| 42 | Titanium dioxide | 13463-67-7 |
| 44 | Zinc oxide | 1314-13-2 |
| 45 | 2,2′-Methylene-bis-[6-(2H-benzotriazol-2-yl)-4-(1,1,3,3-tetramethyl- | 103597-45-1 |
| | butyl)-phenol]; Tinosorb M | |
| 46 | 2,4-bis{[4-(2-ethylhexyloxy)-2-hydroxy]-phenyl}-6-(4- | 187393-00-6 |
| | methoxyphenyl)-(1,3,5)-triazine; Tinosorb S | |
| 47 | 1H-Benzimidazole-4,6-disulfonic acid, 2,2′-(1,4-phenylene)bis-, | 180898-37-7 |
| | disodium salt | |
| 48 | Benzoic acid, 4,4′-[[6-[[4-[[(1,1-dimethylethyl)amino]carbonyl]phenyl]- | 154702-15-5 |
| | amino]1,3,5-triazine-2,4-diyl]diimino]bis-, bis(2-ethylhexyl)ester; di- | |
| | ethylhexyl butamido triazone; Uvasorb HEB | |
| 49 | Phenol, 2-(2H-benzotriazol-2-yl)-4-methyl-6-[2-methyl-3-[1,3,3,3- | 155633-54-8 |
| | tetramethyl-1-[(trimethylsilyl)oxy]disiloxanyl]propyl]-; drometrizole | |
| | trisiloxane; Mexoryl XL | |
| 50 | Dimethicodiethylbenzalmalonate; Polysilicone 15; | 207574-74-1 |
| | Parsol SLX | |
| 51 | Benzenesulfonic acid, 3-(2H-benzotriazol-2-yl)-4-hydroxy-5-(1- | 92484-48-5 |
| | methylpropyl)-, monosodium salt; Tinogard HS | |
| 52 | Benzoic acid, 2-[4-(diethylamino)-2-hydroxybenzoyl]-, hexyl ester; | 302776-68-7 |
| | Uvinul a plus | |
| 53 | 1-Dodecanaminium, N-[3-[[4-(dimethylamino)benzoyl]amino]propyl]- | 156679-41-3 |
| | N,N-dimethyl-, salt with 4-methylbenzenesulfonic acid (1:1); Escalol | |
| | HP610 | |
| 54 | 1-Propanaminium, N,N,N-trimethyl-3-[(1-oxo-3-phenyl-2-propenyl)- | 177190-98-6 |
| | amino]-, chloride | |
| 55 | 1H-Benzimidazole-4,6-disulfonic acid, 2,2′-(1,4-phenylene)bis- | 170864-82-1 |
| 56 | 1,3,5-Triazine, 2,4,6-tris(4-methoxyphenyl)- | 7753-12-0 |
| 57 | 1,3,5-Triazine, 2,4,6-tris[4-[(2-ethylhexyl)oxy]phenyl]- | 208114-14-1 |
| 58 | 1-Propanaminium, 3-[[3-[3-(2H-benzotriazol-2-yl)-5-(1, 1- | 340964-15-0 |
| | dimethylethyl)-4-hydroxyphenyl]-1-oxopropyl]amino]-N,N-diethyl-N- | |
| | methyl-, methyl sulfate (salt) | |
| 59 | 2-Propenoic acid, 3-(1H-imidazol-4-yl)- | 104-98-3 |
| 60 | Benzoic acid, 2-hydroxy-, [4-(1-methylethyl)phenyl]methyl ester | 94134-93-7 |
| 61 | 1,2,3-Propanetriol, 1-(4-aminobenzoate); glyceryl PABA | 136-44-7 |
| 62 | Benzeneacetic acid, 3,4-dimethoxy-a-oxo- | 4732-70-1 |
| 63 | 2-Propenoic acid, 2-cyano-3,3-diphenyl-, ethyl ester | 5232-99-5 |
| 64 | Anthralinic acid, p-menth-3-yl ester | 134-09-8 |
| 65 | 2,2′-bis(1,4-phenylene)-1H-benzimidazole-4,6-disulphonic acid mo- | 349580-12-7, |
| | no sodium salt or Disodium phenyl dibenzimidazole tetrasulfonate or | |
| | Neoheliopan AP | |
| 66 | 1,3,5-Triazine-2,4,6-triamine, N,N′-bis[4-[5-(1,1-dimethylpropyl)-2- | 288254-16-0 |
| | benzoxazolyl]phenyl]-N″-(2-ethylhexyl)- or Uvasorb K2A | |
| 67 | Merocyanine derivatives as described in WO 2004006878 and in | |
| | IPCOM000022279D | |
| 68 | 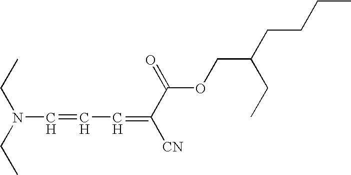 | |
| 68 | sterols (cholesterol, lanosterol, phytosterols), as described in | |
| | WO0341675 | |
| 69 | mycosporines and/or mycosporine-like amino acids as described in | |
| | WO2002039974, e.g. Helioguard 365 from Milbelle AG, isolated | |
| | mycosporine like amino acids from the red alga porphyra umbilicalis | |
| | (INCI: Porphyra Umbilicalis) that are encapsulated into liposomes,) | |
| 70 | alpha-lipoic-acid as described in DE 10229995 | |
| 71 | synthetic organic polymers as described in EP 1371358, [0033]- | |
| | [0041] | |
| 72 | phyllosilicates as described in EP 1371357 [0034]-[0037] | |
| 73 | silica compounds as described in EP 1371356, [0033]-[0041] | |
| 74 | inorganic particles as described in DE10138496 [0043]-[0055] | |
| 75 | latex particles as described in DE10138496 [0027]-[0040] | |
| 76 | 1H-Benzimidazole-4,6-disulfonic acid, 2,2′-(1,4-phenylene)bis-, | 180898-37-7 |
| | disodium salt; Bisimidazylate; Neo Heliopan APC | |

Suitable UV filter substances which can be additionally used

(Abbreviations T: table, R: row, Comp: compound, Ex: compound(s) of patent example, p:
page); the generic scope of the UV absorbers is described in the left-hand column; specific
compounds are indicated in the right-hand column

| | |
|---|---|
| DE 100331804 | Tab 1 p 4, tab 2 + 3 p 5 |
| EP 613893 | Ex 1-5 + 15, T 1, pp 6-8 |
| EP 1000950 | Comp. in table 1, pp 18-21 |
| EP 1005855 | T 3, p 13 |
| EP 1008586 | Ex 1-3, pp 13-15 |
| EP 1008593 | Ex 1-8, pp 4-5 |
| EP 1027883 | Compound VII, p 3 |
| EP 1027883 | Comp 1-VI, p 3 |
| EP 1028120 | Ex 1-5, pp 5-13 |
| EP 1059082 | Ex 1; T 1, pp 9-11 |
| EP 1060734 | T 1-3, pp 11-14 |
| EP 1064922 | Compounds 1-34, pp 6-14 |
| EP 1081140 | Ex 1-9, pp 11-16 |
| EP 1103549 | Compounds 1-76, pp 39-51 |
| EP 1108712 | 4,5-Dimorpholino-3-hydroxypyridazine |
| EP 1123934 | T 3, p 10 |
| EP 1129695 | Ex 1-7, pp 13-14 |
| EP 1167359 | Ex 1 p 11 and ex 2 p 12 |
| EP 1258481 | Ex 1, pp 7, 8 |
| EP 420707 B1 | Ex 3, p 13 (CAS Regno 80142-49-0) |
| EP 503338 | T1, pp 9-10 |
| EP 517103 | Ex 3, 4, 9, 10 pp 6-7 |
| EP 517104 | Ex 1, T 1, pp 4-5; Ex 8, T 2, pp 6-8 |
| EP 626950 | all compounds |
| EP 669323 | Ex 1-3, p 5 |
| EP 780382 | Ex 1-11, pp 5-7 |
| EP 823418 | Ex 1-4, pp 7-8 |
| EP 826361 | T 1, pp 5-6 |
| EP 832641 | Ex 5 + 6 p 7; t 2, p 8 |
| EP 832642 | Ex 22, T 3 pp, 10-15; T 4, p 16 |
| EP 852137 | T 2, pp 41-46 |
| EP 858318 | T 1, p 6 |
| EP 863145 | Ex 1-11, pp 12-18 |
| EP 895776 | Comp. in rows 48-58, p 3; R 25 + 33, p 5 |
| EP 911020 | T 2, p 11-12 |
| EP 916335 | T 2-4, pp 19-41 |
| EP 924246 | T 2, p 9 |
| EP 933376 | Ex 1-15, pp 10-21 |
| EP 944624 | Ex 1 + 2, pp 13-15 |
| EP 945125 | T 3 a + b, pp 14-15 |
| EP 967200 | Ex 2; T 3-5, pp 17-20 |
| EP 969004 | Ex 5, T 1, pp 6-8 |
| JP 2000319629 | CAS Regno. 80142-49-0, |
| | 137215-83-9, 307947-82-6 |
| US 5635343 | all compounds on pp 5-10 |
| US 5338539 | Ex 1-9, pp 3 + 4 |
| US 5346691 | Ex 40, p 7; T 5, p 8 |
| US 5801244 | Ex 1-5, pp 6-7 |
| WO 0149686 | Ex 1-5, pp 16-21 |
| WO 0168047 | Tables on pp 85-96 |
| WO 0181297 | Ex 1-3 pp 9-11 |
| WO 0238537 | All componds p 3, compounds on rows 1-10 p 4 |
| WO 9217461 | Ex 1-22, pp 10-20 |
| WO 9220690 | Polymeric comp in examples 3-6 |
| WO 9301164 | T 1 + 2, pp 13-22 |
| WO 9714680 | Ex 1-3, p 10 |

As water- and oil-containing emulsions (e.g. W/O, O/W, O/W/O and W/O/W emulsions or microemulsions) the preparations contain, for example, from 0.1 to 30% by weight, preferably from 0.1 to 15% by weight and especially from 0.5 to 10% by weight, based on the total weight of the composition, of one or more UV absorbers, from 1 to 60% by weight, especially from 5 to 50% by weight and preferably from 10 to 35% by weight, based on the total weight of the composition, of at least one oil component, from 0 to 30% by weight, especially from 1 to 30% by weight and preferably from 4 to 20% by weight, based on the total weight of the composition, of at least one emulsifier, from 10 to 90% by weight, especially from 30 to 90% by weight, based on the total weight of the composition, of water, and from 0 to 88.9% by weight, especially from 1 to 50% by weight, of further cosmetically acceptable adjuvants.

Cosmetic or pharmaceutical preparations in general can be prepared by physically mixing the active component(s) with the adjuvant using customary methods, for example by simply stirring together the individual components.

Examples for compounds especially useful in the present invention include those listed below:

AO3: Reaction product of glycerine, coconut oil and a compound of the formula

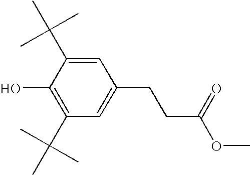

(mixture of mono-, di- and triglycerides of β-(3,5-di-tert.butyl-4-hydroxyphenyl) propanoic acid and fatty acids, CAS Reg.-No. 179986-09-5)

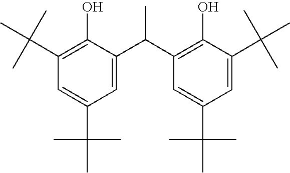

### AO4:

AO5 is a mixture of the compounds of the formulae:

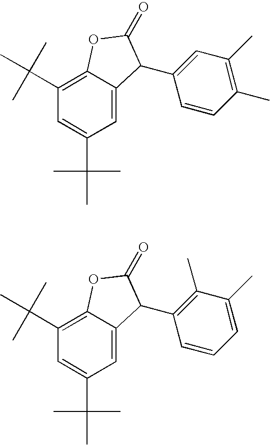

### AO6:

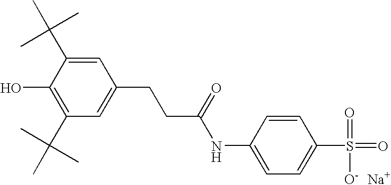

AO7: pentaerythritol-tetrakis(3-[3′,5′-di-tert.butyl-4′-hydroxyphenyl]-propionate)

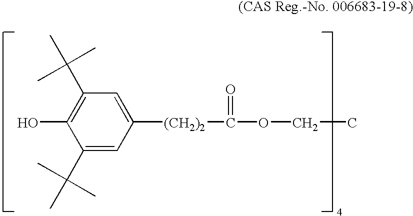

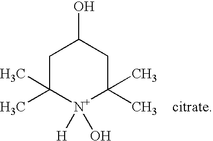

### AO8:

The following Examples will serve to illustrate the invention without implying any restriction to what is described therein. Unless otherwise indicated, percentages are by weight (% w/w; % b.w.). The abbreviation “qs” denotes a percentage adjustable to fulfill product specifications; e.g. fragrance: 0.1-0.2%; colorant 0.05-0.2%.

### Example 1

### Dermatological Formulations

### General Preparation:

A clear homogenous prephase is obtained by mixing

(a) 5 to 20% by weight of a phospholipid (e.g. lecithin),
(b) 15 to 40% by weight of a coemulsifier (e.g. a polyethoxylated sorbitan fatty acid ester, polyethoxylated fatty alcohol, polyethoxylated fatty acid, polyethoxylated vitamin E derivative, polyethoxylated lanoline or lanoline derivative, polyethoxylated fatty acid glyceride or partial glyceride, polyethoxylated alkylphenol, sulfuric acid semiester of a polyethoxylated fatty alcohol or salt thereof, polyethoxylated fatty amine or amide, polyethoxylated carbon hydrate),
(c) 30 to 70% by weight of a lipophilic component consisting of a triglyceride, a compound of the invention such as AO3-AO8, and optionally a further active agent, where the weight ratio of active agent(s):triglyceride usually ranges from 1:5 to 5:1, and
(d) 3 to 30% by weight of an alcohol such as ethanol,
with the sum of percentages of the components (a), (b), (c) and (d) adding to 100%,
and
adding the liquid obtained to a water phase. The water phase (e.g. 90 kg) is placed, with stirring (e.g. magnetic agitator), at 50° C. in a vessel. The liquid prephase (e.g. 10 kg) is added to the water phase with stirring (e.g. with a magnetic agitator). The formulation thus obtained may be further diluted or admixed.

### Vitamin A Palmitate Cream

A dispersion is obtained by mixing a clear prephase containing:

| | |
|---|---|
| vitamin A palmitate (1.7 × 10⁶ IU/g) | 0.45% |
| compound AO7 | 0.45% |
| soybean lecithin | 1.73% |
| miglyol 812 | 3.00% |
| polysorbate 80 | 3.40% |
| ethanol | 1.42% |
| to a water phase: | |
| 10 mm phosphate buffer, pH 6 | ad 100.00% |
| Final preparation contains: | |
| cetyl alcohol | 10.00% |
| hydrogenated groundnut oil | 20.00% |
| polysorbate 60 | 5.00% |
| propylene glycol | 20.00% |
| phenoxyethanol | 0.50% |
| dispersion shown above | 23.00% |
| aqua purificata | ad 100.00% |

Good results are also achieved when compound AO7 is replaced by AO3, AO4 or AO5.

### Solcoseryl 0.5% Hydrogel

A dispersion is obtained by mixing a clear prephase containing:

| | |
|---|---|
| solcoseryl | 1.00% |
| compound AO7 | 1.00% |
| soybean lecithin | 1.73% |
| polysorbate 80 | 3.40% |
| miglyol 812 | 3.45% |
| ethanol | 1.42% |
| to a water phase: | |
| 10 mm phosphate buffer, pH 6 | ad 100.00% |
| Final preparation contains: | |
| sodium carboxymethylcellulose 450 cP | 3.50% |
| dispersion shown above | 50.00% |
| aqua purificata | ad 100.00% |

The preparation is pleasantly cooling and has good antiphlogistic action.

### Skin Protecting W/O Lotion

A dispersion is obtained by mixing a clear prephase containing:

| | |
|---|---|
| vitamin E acetate | 2.00% |
| compound AO3 | 1.00% |
| soybean lecithin | 0.49% |
| polysorbate 80 | 1.86% |
| miglyol 812 | 0.71% |
| ethanol | 0.63% |
| to a water phase: | |
| aqua purificata | ad 100.00% |
| Final preparation contains: | |
| glycerol sorbitan fatty acid ester | 2.0% |
| polyethoxy fatty acid ester | 2.0% |
| isopropylisostearate | 5.0% |
| mineral oil | 7.0% |
| isopropylpalmitate | 4.0% |
| wheat germ oil | 3.0% |
| propylene glycol | 3.8% |
| dispersion shown above | 5.0% |
| MgSO₄ × 7H2O | 0.7% |
| perfume | 0.5% |
| perservative, water | |

Good results are also achieved when compound AO3 is replaced by AO7, AO6 or AO8.

Day Cream with UV Protection (O/W)

A dispersion is obtained by mixing a clear prephase containing:

| | |
|---|---|
| parsol MCX | 2.59% |
| (octyl methoxycinnamate) | |
| Parsol 5000 | 1.11% |
| (4-methylbenzylidene camphor) | |
| compound AO7 | 1.00% |
| miglyol 812 | 1.30% |
| soybean lecithin | 0.50% |
| polysorbate 80 | 3.40% |
| ethanol | 1.10% |
| to a water phase: | |
| aqua purificata | ad 100.00% |
| Final preparation contains: | |
| PEG-5 glycerol stearate | 5.0% |
| steareth-21 | 2.0% |
| mineral oil | 30.0% |
| cetyl alcohol | 2.0% |
| microcrystalline wax | 1.0% |
| propylene glycol | 6.0% |
| dispersion shown above | 10.0% |
| phenoxyethanol + methyl-, ethyl-, propyl-, butylparabene | 0.3% |
| water | ad 100.0% |

### Stabilized Vitamin C

A) Coencapsulation Together with the Antioxidant:

A dispersion is obtained by mixing a clear prephase containing:

| | |
|---|---|
| ascorbic acid | 1.00% |
| compound AO7 | 1.00% |
| soybean lecithin | 1.73% |
| polysorbate 80 | 3.40% |
| miglyol 812 | 3.45% |
| ethanol | 1.42% |
| to a water phase: | |
| 10 mm phosphate buffer, pH 6 | ad 100.00% |

### B) Stabilization of Free Vitamin C by Encapsulated Nanodispersed Antioxidant:

A dispersion is obtained by mixing a clear prephase containing:

| | |
|---|---|
| compound AO7 | 1.00% |
| soybean lecithin | 1.73% |
| polysorbate 80 | 3.40% |
| miglyol 812 | 4.45% |
| ethanol | 1.42% |
| to a water phase: | |
| ascorbic acid | 1.00% |
| 10 mm phosphate buffer, pH 6 | ad 100.00% |

C) Coencapsulation Together with the Antioxidant Plus Free Antioxidant:

A dispersion is obtained by mixing a clear prephase containing:

| | |
|---|---|
| ascorbic acid | 1.00% |
| compound AO7 | 0.50% |
| soybean lecithin | 1.73% |
| polysorbate 80 | 3.40% |
| miglyol 812 | 3.45% |
| ethanol | 1.42% |
| to a water phase: | |
| compound AO6 | 0.50% |
| 10 mm phosphate buffer, pH 6 | ad 100.00% |

### D) Stabilization of Free Vitamin C by Encapsulated Nanodispersed Plus Free Antioxidant:

A dispersion is obtained by mixing a clear prephase containing:

| | |
|---|---|
| compound AO7 | 0.50% |
| soybean lecithin | 1.73% |
| polysorbate 80 | 3.40% |
| miglyol 812 | 4.45% |
| ethanol | 1.42% |
| to a water phase: | |
| ascorbic acid | 1.00% |
| compound AO6 | 0.50% |
| 10 mm phosphate buffer, pH 6 | ad 100.00% |

Concentrated dispersions of high stability are thus obtained.

The final preparations based on concentrations A), B), C) or D) contain:

| | |
|---|---|
| sodium carboxymethylcellulose 450 cP | 3.50% |
| dispersion shown above | 10.00% |
| aqua purificata | ad 100.00% |

Good results are also achieved when compound AO7 is replaced by AO3, AO4 or AO5.

### Example 2

### Stabilization of Vitamin C

### Materials:

Aqueous solution of Vitamin C (concentration=40.5 mM), buffered at pH=6 (Phosphate buffer, previously evacuated and then flushed with Nitrogen to remove as much Oxygen as possible).

### Solutions:

### a) Standalone Vitamin C: 0.7 mM

### b) Combination Vitamin C/Nanotope AO7:

A dispersion is obtained by mixing a clear prephase containing:

| | |
|---|---|
| compound AO7 | 1.00% |
| soybean lecithin | 1.73% |
| polysorbate 80 | 3.40% |
| miglyol 812 | 4.45% |
| ethanol | 1.42% |
| to a water phase: | |
| ascorbic acid | 1.00% |
| 10 mm phosphate buffer, pH 6 | ad 100.00% |

### Apparatus:

Absorption measurement with Perkin Elmer Lambda 16; cuvette length d=0.1 cm Irradiation chamber Atlas CPS+; temperature of measurement=35° C.

### Method:

Spectral absorption of Vitamin C (Ascorbic Acid) at its maximum wavelength (Iₘₐₓ=265 nm); maximum extinction at time after exposure to air with storage in the dark. Experiments were done at 20° C.

Vitamin C is efficiently protected by the encapsulated antioxidant.

### Example 3

### Coencapsulation of Active Ingredients

### a) General Preparation:

A nanodispersion prephase is prepared by mixing the following components:

| | Combination Sample No. | | | | | | | |
|---|---|---|---|---|---|---|---|---|
| | 1 | 2 | 3 | 4 | 5 | 6 | 7 | 8 |
| Component: | parts by weight: | | | | | | | |
| antioxidant (such as AO3, | 0.1 | 0.5 | 1 | 3 | 5 | 7 | | 10 |
| AO4, AO5, AO6, AO7 | | | | | | | | |
| and/or AO8) | | | | | | | | |
| Soybean Lecithin | 10 | 5 | 15 | 20 | 10 | 18 | 8 | 18 |
| Miglyol 812 | 60 | 35 | 45 | 60 | 55 | 40 | 30 | 50 |
| Polysorbate 80 | 27 | 35 | 20 | 10 | 15 | 30 | 39 | 13 |
| Ethanol | 3 | 20 | 17 | 7 | 10 | 4 | 12 | 4 |
| Active ingredient | 0.5 | 5 | 2 | — | 5 | 1 | 3 | 5 |
| (such as vitamin A, C, E) | | | | | | | | |

The nanodispersion (concentrate) is generated by combining 5% to 50% b.w., and most preferably from 10% to 30% b.w., of the prephase to deionized water (ad 100%) with stirring.

The final formulation is then obtained using from 0.1% to 90%, usually from 0.1% to 20%, of the concentrate (based on the total weight of the cosmetic or pharmaceutic application).

### b) Formulations Ready for End Use:

Each formulation listed below is obtained by mixing the parts indicated in a table.

TABLE 3.1
UV-A/UV-B Daily Care UV Protection Lotion

| | | % w/w (as supplied) in formulation No.: | | | | | |
|---|---|---|---|---|---|---|---|
| | INCI-Name | 4a | 4b | 4c | 4d | 4e | 4f |
| Part A | Oleth-3 Phosphate | 0.60 | 0.60 | 0.60 | 0.60 | 0.60 | 0.60 |
| | Steareth-21 | 2.50 | 2.50 | 2.50 | 2.50 | 2.50 | 2.50 |
| | Steareth-2 | 1.00 | 1.00 | 1.00 | 1.00 | 1.00 | 1.00 |
| | Cetyl Alcohol | 0.80 | 0.80 | 0.80 | 0.80 | 0.80 | 0.80 |
| | Stearyl Alcohol | 1.50 | 1.50 | 1.50 | 1.50 | 1.50 | 1.50 |
| | Tribehenin | 0.80 | 0.80 | 0.80 | 0.80 | 0.80 | 0.80 |
| | Isohexadecane | 8.00 | 8.00 | 8.00 | 8.00 | 8.00 | 8.00 |
| | Ethylhexyl Methoxycinnamate | 3.00 | 3.00 | 3.00 | 3.00 | 3.00 | 3.00 |
| | Bis-ethylhexyloxyphenol | 2.00 | — | 1.00 | 2.00 | 1.50 | 2.00 |
| | methoxyphenyl triazine | | | | | | |
| | Diethylamino Hydroxybenzoyl | — | 1.30 | — | — | — | — |
| | Hexyl Benzoate | | | | | | |
| | Ethylhexyl Triazone | — | — | 1.50 | — | — | — |
| | Diethylhexyl butamido triazone | — | — | — | 2.00 | — | — |
| | Butyl methoxydibenzoylmethane | — | — | — | — | 1.00 | |
| | Benzylidene malonate | — | — | — | — | — | 2.00 |
| | polysiloxane | | | | | | |
| Part B | Water | ad 100 | ad 100 | ad 100 | ad 100 | ad 100 | ad 100 |
| | Glycerin | 2.00 | 2.00 | 2.00 | 2.00 | 2.00 | 2.00 |
| | Methylene bis-benzotriazolyl | 3.00 | 2.00 | 1.50 | 1.00 | | 4.00 |
| | tetramethylbutylphenol | | | | | | |
| | Disodium Phenyl dibenzimidazole | 1.00 | 1.00 | 2.00 | — | 3.00 | — |
| | Tetrasulfonate | | | | | | |
| | Disodium EDTA | 0.10 | 0.10 | 0.10 | 0.10 | 0.10 | 0.10 |
| Part C | Water | 20.00 | 20.00 | 20.00 | 20.00 | 20.00 | 20.00 |
| | Diazolidinyl Urea (and) | 0.15 | 0.15 | 0.15 | 0.15 | 0.15 | 0.15 |
| | Iodopropynyl Butylcarbamate | | | | | | |
| | Propylene Glycol | 4.00 | 4.00 | 4.00 | 4.00 | 4.00 | 4.00 |
| Part D | Sodium Acrylates Copolymer | 1.50 | 1.50 | 1.50 | 1.50 | 1.50 | 1.50 |
| | (and) Paraffinium Liquidum (and) | | | | | | |
| | PPG-1 Trideceth-6 | | | | | | |
| | Cyclopentasiloxane | 4.00 | 4.00 | 4.00 | 4.00 | 4.00 | 4.00 |
| | PEG-12 Dimethicone | 2.00 | 2.00 | 2.00 | 2.00 | 2.00 | 2.00 |
| | Tocopheryl Acetate | 0.45 | 0.45 | 0.45 | 0.45 | 0.45 | 0.45 |
| | aqueous Citric Acid | * | * | * | * | * | * |
| Part E | Nanodispersion according to the | 1.00 | 3.00 | 5.00 | 7.00 | 10.00 | 20.00 |
| | invention; combination from1 to 8 | | | | | | |
| | Vitamin A, C or E (free or | 0.80 | 0.50 | — | 1.00 | — | — |
| | derivative form) | | | | | | |
| | Fragrance | qs | qs | qs | qs | qs | qs |

* aqu. citric acid to pH 6-pH 7

TABLE 3.2
Sprayable Sunscreen Emulsion

| | | % w/w (as supplied) in formulation No. | | | |
|---|---|---|---|---|---|
| | INCI-Name | 5a | 5b | 5c | 5d |
| Part A | Ceteareth-15 (and) Glyceryl Stearate | 3.00 | 3.00 | 3.00 | 3.00 |
| | Stearyl Alcohol | 1.00 | 1.00 | 1.00 | 1.00 |
| | Cetyl Ricinoleate | 0.80 | 0.80 | 0.80 | 0.80 |
| | Dicaprylyl Ether | 3.00 | 3.00 | 3.00 | 3.00 |
| | C12-15 Alkyl Benzoate | 3.00 | 3.00 | 3.00 | 3.00 |
| | Isohexadecane | 2.50 | 2.50 | 2.50 | 2.50 |
| | Stearyl Dimethicone | 1.00 | 1.00 | 1.00 | 1.00 |
| | Ethylhexyl Methoxycinnamate | 4.00 | 4.00 | 4.00 | 4.00 |
| | Cetyl Alcohol | 0.80 | 0.80 | 0.80 | 0.80 |
| | Di-C12-13 Alkyl Tartrate | 3.00 | 3.00 | 3.00 | 3.00 |
| Part B | Aqua | qs to 100 | qs to 100 | qs to 100 | qs to 100 |
| | Steareth-10 Allyl Ether/Acrylates Copolymer | 0.45 | 0.45 | 0.45 | 0.45 |
| | PEG-7 Glyceryl Cocoate | 2.50 | 2.50 | 2.50 | 2.50 |
| | Glycerin | 2.00 | 2.00 | 2.00 | 2.00 |
| | Propylene Glycol | 3.00 | 3.00 | 3.00 | 3.00 |
| Part C | Diazolidinyl Urea (and) Iodopropynyl | 0.15 | 0.15 | 0.15 | 0.15 |
| | Butylcarbamate | | | | |
| | Aqua | 20.00 | 20.00 | 20.00 | 20.00 |
| | Methylene bis-benzotriazolyl | 3.00 | 2.00 | 1.00 | 4.00 |
| | tetramethylbutylphenol (TinosorbM) | | | | |
| | Titanium Dioxide (and) Silica (and) Sodium | 8.00 | 5.00 | 3.00 | 2.00 |
| | Polyacrylate | | | | |
| Part D | Cyclopentasiloxane (and) Dimethiconol | 0.85 | 0.85 | 0.85 | 0.85 |
| Part E | aqueous Sodium Hydroxide | * | * | * | * |
| Part F | Nanodispersion according to the invention; | 1.00 | 3.00 | 5.00 | 7.00 |
| | combination from1 to 8 | | | | |
| | Vitamins A, C or E (free or derivative forms) | 0.80 | 0.50 | — | 1.00 |
| | Fragrance | qs | qs | qs | qs |

* Sodium Hydroxide and Water: to pH 6.50-7.00

TABLE 3.3
O/W Every Day UV Protection Lotion

| | | % w/w (as suppl.), form. No. | | | |
|---|---|---|---|---|---|
| | INCI-Name | 8a | 8b | 8c | 8d |
| Part A | Glyceryl Stearate (and) PEG-100 Stearate | 5.00 | 5.00 | 5.00 | 5.00 |
| | Stearyl Alcohol | 1.00 | 1.00 | 1.00 | 1.00 |
| | Tripalmitin | 0.70 | 0.70 | 0.70 | 0.70 |
| | Dimethicone | 2.00 | 2.00 | 2.00 | 2.00 |
| | C12-15 Alkyl Benzoate | 5.00 | 5.00 | 5.00 | 5.00 |
| | Isopropyl Palmitate | 5.00 | 5.00 | 5.00 | 5.00 |
| | Ethylhexyl Methoxycinnamate | 3.00 | 3.00 | 3.00 | 3.00 |
| Part B | Water | ad 100 | ad 100 | ad 100 | ad 100 |
| | Polysorbate 60 | 0.50 | 0.50 | 0.50 | 0.50 |
| | Glycerin | 3.00 | 3.00 | 3.00 | 3.00 |
| Part C | Water | 10.00 | 10.00 | 10.00 | 10.00 |
| | Methylene bis-benzotriazolyl | 4.00 | 3.00 | 2.00 | 1.00 |
| | tetramethylbutylphenol (TinosorbM) | | | | |
| Part D | Phenoxyethanol (and) Methylparaben (and) | 0.70 | 0.70 | 0.70 | 0.70 |
| | Ethylparaben (and) Butylparaben (and) | | | | |
| | Propylparaben (and) Isobutylparaben | | | | |
| | Steareth-10 Allyl Ether/Acrylates Copolymer | 1.50 | 1.50 | 1.50 | 1.50 |
| Part E | aq. Sodium Hydroxide | * | * | * | * |
| Part F | Nanodispersion according to the invention; | 1.00 | 3.00 | 5.00 | 7.00 |
| | combination from1 to 8 | | | | |
| | Vitamins A, C or E (free or derivative forms) | 0.80 | 0.50 | — | 1.00 |
| | Fragrance | qs | qs | qs | qs |

* aq. Sodium Hydroxide to pH 6.5-7

TABLE 3.4
W/O Emulsions

| | Formulation No. [% b.w.] | | | | | | | |
|---|---|---|---|---|---|---|---|---|
| Component | 10a | 10b | 10c | 10d | 10e | 10f | 10g | 10h |
| Polyglyceryl-3 Diisostearate | 6.0 | — | — | — | — | — | — | — |
| Polyglyceryl-2 | — | 5.0 | — | 6.0 | — | — | 3.0 | — |
| Dipolyhydroxystearate | | | | | | | | |
| PEG-30 Dipolyhydroxystearate | — | — | 5.5 | — | — | — | — | 4.0 |
| Cetyl Dimethicone Copolyol | — | — | — | — | 5.0 | 1.5 | 4.0 | — |
| Laurylmethicone Copolyol | — | — | — | — | — | 4.0 | — | 2.5 |
| Methylene bis-benzotriazolyl | 5.0 | 0.5 | 3.0 | 5.0 | 2.0 | 2.5 | 3.5 | 2.5 |
| tetramethylbutylphenol | | | | | | | | |
| (TinosorbM) | | | | | | | | |
| Aniso Triazine | — | 2.0 | 0.5 | — | — | — | — | 4.5 |
| Dioctyl Butamidotriazone | — | 1.0 | 2.5 | — | — | 2.0 | — | — |
| Ethylhexyl Triazone | — | 2.0 | — | — | 4.0 | — | — | — |
| Bisoctyltriazol | 1.5 | — | — | 4.0 | — | — | — | — |
| Drometrizol Trisiloxane | 2.0 | — | 3.0 | — | 4.0 | — | — | 5.0 |
| Phenylbenzmidazole Sulfonic Acid | | 1.0 | — | — | 2.0 | — | — | — |
| Bisimidazylate | — | — | — | 2.5 | — | 2.0 | 2.0 | — |
| Terephthalylidene Dicamphor | 0.75 | — | — | — | — | — | — | 1.0 |
| Sulfonic Acid | | | | | | | | |
| Ethylhexyl Methoxycinnamate | — | 7.5 | 5.0 | — | — | — | — | 8.0 |
| Octocrylene | — | — | 5.0 | — | — | 10.0 | — | 4.0 |
| Dimethicone-diethylbenzal- | 7.0 | — | — | — | 2.5 | — | — | — |
| malonate | | | | | | | | |
| Ethylhexyl Salicylate | — | — | 5.0 | — | — | — | — | 4.0 |
| Homosalate | — | — | 3.5 | — | — | — | — | — |
| Butyl Methoxydibenzoylmethane | 1.5 | — | — | — | 0.5 | — | — | — |
| 4-Methylbenzylidene Camphor | 3.0 | — | — | — | — | — | — | — |
| Micronised Titanium Dioxide | — | 3.0 | 6.0 | — | — | 2.0 | 2.0 | 3.0 |
| Micronised Zinc Oxide | — | — | — | — | 8.0 | 7.0 | — | — |
| Paraffine Oil | 20.0 | 15.0 | — | 10.0 | — | — | 15.0 | 10.0 |
| Vaseline | — | 2.0 | — | 5.0 | — | — | — | — |
| Cyclomethicone | — | — | — | — | 25.0 | — | 10.0 | — |
| Dimethicone | — | — | 4.0 | — | 10.0 | 3.0 | — | — |
| Dicaprylylcarbonate | 10.0 | — | 9.0 | — | — | — | 10.0 | — |
| C12-C15 Alkyl Benzoate | 5.0 | — | — | 10.0 | — | 9.0 | — | — |
| Butylen Glycol | — | 10.0 | — | — | — | 10.0 | — | 3.0 |
| Dicaprylat/eDicaprate | | | | | | | | |
| Octyldodecanol | — | — | 10.0 | 15.0 | — | — | 5.0 | — |
| MgSO₄ | 0.7 | 0.5 | — | 0.4 | 1.0 | 1.0 | — | 1.5 |
| Glycerine | — | 10.0 | 5.0 | 7.5 | 7.5 | — | 3.0 | — |
| Parfume/Fragrance | 0.45 | 0.2 | 0.3 | — | — | 0.4 | — | 0.2 |
| Ethanol | — | 3.5 | — | — | 4.0 | — | 5.0 | 4.0 |
| Octoxyglycerine | — | — | 0.5 | — | — | — | 1.0 | — |
| Tocopherol or Tocopherolacetate | 0.5 | 0.75 | — | 0.3 | — | 0.5 | — | 1.0 |
| Trisodium EDTA | 0.1 | 0.2 | — | 0.1 | — | 0.3 | — | 0.1 |
| Phenonip ® | — | 0.4 | 0.5 | — | 1.0 | 0.5 | — | — |
| DMDM Hydantoine | 0.1 | — | — | 0.2 | 0.05 | — | — | — |
| Water | ad 100 | ad 100 | ad 100 | ad 100 | ad 100 | ad 100 | ad 100 | ad 100 |
| Nanodispersion according to the | 1.00 | 2.00 | 3.00 | 5.00 | 7.00 | 8.00 | 10.00 | 20.00 |
| invention; combination from1 to 8 | | | | | | | | |
| Vitamin A, C or E (free or | 0.80 | 0.50 | — | — | 2.00 | 1.00 | — | 1.50 |
| derivative forms) | | | | | | | | |

TABLE 3.4
Hydrodispersions

| | Formulation No. [% b.w.] | | | | |
|---|---|---|---|---|---|
| Component | a | b | c | d | e |
| Polyoxyethylene(20)cetylstearylether | 1.00 | — | — | 0.5 | — |
| Cetylalcohol | — | — | 1.00 | — | — |
| Natriumpolyacrylate | — | 0.20 | — | 0.30 | — |
| Acrylate/C10-30-Alkyl-Acrylate Crosspolymer | 0.50 | | 0.40 | 0.10 | 0.10 |
| Xanthan Gum | — | 0.30 | 0.15 | — | 0.50 |
| 2-Ethylhexyl Methoxycinnamate | — | — | — | 5.00 | 8.00 |
| 2,4-bis{[4-(2-ethylhexyloxy)-2-hydroxy]-phenyl}- | — | 1.50 | 2.00 | — | 2.50 |
| 6-(4-methoxyphenyl)-(1,3,5)-triazine | | | | | |
| (Tinosorb ® S) | | | | | |
| Dimethicodiethylbenzalmalonate | — | 3.50 | — | — | — |
| Butyl-dimethoxy-dibenzoylmethane | 1.00 | — | 2.00 | — | — |
| Diethylhexyl Butamidotriazone | — | 2.00 | — | 2.00 | 1.00 |
| Ethylhexyl Triazone | 4.00 | — | 3.00 | 4.00 | — |
| 4-Methylbenzylidene Camphor | — | — | — | — | 2.00 |
| Methylene bis-benzotriazolyl | 2.00 | 1.40 | 0.50 | 4.60 | 5.20 |
| tetramethylbutylphenol (Tinosorb ® M) | | | | | |
| Octocrylene | — | 4.00 | 4.00 | — | 2.50 |
| Phenylen-1,4-bis-(monosodium-2- | 1.00 | — | 0.50 | — | 2.00 |
| benzimidazyl-5,7-disulfonic Acid) | | | | | |
| Phenylbenzimidazol Sulfonic Acid | 0.50 | — | — | 3.00 | — |
| Titanium Dioxide | 0.50 | — | 2.00 | 3.00 | 1.00 |
| Zinc Oxide | 0.50 | 1.00 | 3.00 | — | 2.00 |
| C12-15 Alkyl Benzoate | 2.00 | 2.50 | — | — | — |
| Diethylhexyl-2.6-naphthalate | 4.00 | — | — | — | — |
| Dicaprylylether | — | 4.00 | — | — | — |
| Butylenglycol-Dicaprylate/-Dicaprate | 4.00 | — | 2.00 | 6.00 | — |
| Dicaprylylcarbonate | — | 2.00 | 6.00 | — | — |
| Dimethicone Polydimethylsiloxane | — | 0.50 | 1.00 | — | — |
| Phenylmethylpolysiloxane | 2.00 | — | — | 0.50 | 2.00 |
| Shea Butter | — | 2.00 | — | — | — |
| PVP Hexadecenecopolymer | 0.50 | — | — | 0.50 | 1.00 |
| Octoxyglycerine | — | — | 1.00 | — | 0.50 |
| Glycerine | 3.00 | 7.50 | — | 7.50 | 2.50 |
| Glycinesoja | — | — | 1.50 | — | — |
| Dioic Acid | 0.3 | 0.10 | 0.50 | 1.00 | 0.20 |
| DMDM Hydantoine | — | 0.25 | — | 0.60 | 0.45 |
| Parabene | 0.15 | — | 0.50 | 0.30 | — |
| Trisodium EDTA | — | — | 0.70 | — | 1.00 |
| Phenoxyethanol | — | 0.40 | — | 0.20 | 0.50 |
| Ethanol | 3.00 | 2.00 | 1.50 | — | 1.00 |
| Water | ad. 100 | ad. 100 | ad. 100 | ad. 100 | ad. 100 |
| Nanodispersion according to the invention; | 1.00 | 3.00 | 5.00 | 7.00 | 10.00 |
| combination from1 to 8 | | | | | |
| Vitamins A, C or E (free or derivative forms) | 0.80 | 0.50 | — | 1.00 | — |
| Fragrance | qs | qs | qs | qs | qs |

TABLE 3.5
Pickering Emulsions

| | Examples | | | |
|---|---|---|---|---|
| Component [% b.w.] | a | b | c | d |
| Octyldodecanol | 5.50 | 5.50 | 5.00 | 5.50 |
| C12-15-Alkyl Benzoate | 6.50 | 6.50 | 6.00 | 6.50 |
| Butylene Glycol Caprylate/Caprate | — | — | 4.50 | — |
| Dicaprylyl Ether | 5.50 | 5.50 | 3.50 | 5.50 |
| Hydroxyoctacosanyl | 2.00 | 2.00 | 2.50 | 2.00 |
| Hydroxystearate | | | | |
| Disteardimonium Hectorite | 1.00 | 1.00 | 0.50 | 1.00 |
| Butyl Methoxydibenzoylmethane | — | — | 1.00 | — |
| Methylbenzylidene Camphor | — | — | 2.00 | — |
| Ethylhexyltriazone | — | — | 1.00 | — |
| 2.2′-Methylene-bis-(6-(2H- | 2.00 | 1.00 | — | 4.00 |
| benzotriazole-2-yl)-4-(1.1.3.3- | | | | |
| tetramethylbutyl)-phenol | | | | |
| (Tinosorb ® M) | | | | |
| Titanium Dioxide + Alumina + | — | — | 0.50 | — |
| Simethicone + Aqua | | | | |
| Titanium Dioxide + | 4.00 | 4.00 | 2.00 | 4.00 |
| Tdmethoxycaprylylsilane | | | | |
| Zinc Oxide | 2.00 | 2.00 | — | 2.00 |
| Bariumsulfate | 0.50 | 0.50 | — | 0.50 |
| Boron Nitride | 2.00 | 2.00 | 1.50 | 2.00 |
| NaCl | 1.00 | 1.00 | — | 1.00 |
| MgSO₄ | — | — | 0.70 | — |
| DHA | — | — | 5.00 | — |
| Glycerine | 10.00 | 10.00- | 5.00 | 10.00 |
| Trisodium EDTA | 1.00 | 1.00 | 1.00 | 1.00 |
| Porpylene Carbonate | 0.33 | 0.33 | — | 0.33 |
| Repellent 3535 | — | — | 2.00 | — |
| Methylparabene | 0.20. | 0.20. | 0.30 | 0.20. |
| Propylparabene | — | — | 0.07 | — |
| Phenoxyethanol | 0.50 | 0.50 | 0.50 | 0.50 |
| Hexamidine Diisethionate | 0.08 | 0.08 | 0.08 | 0.08 |
| Water | ad 100 | ad 100 | ad 100 | ad 100 |
| Nanodispersion according | 1.00 | 3.00 | 5.00 | 7.00 |
| to the invention; combination | | | | |
| from1 to 8 | | | | |
| Vitamin A, C or E (free or | 0.80 | 0.50 | — | 1.00 |
| derivative form) | | | | |
| Fragrance | qs | qs | qs | qs |

TABLE 3.6
W/ Si emulsion

| | | a | b | c | d |
|---|---|---|---|---|---|
| Part | Ingredient | % b.w. | | | |
| A | Masil ® 756 CG (BASF) (tetrabutoxypropyl | 0.90 | 0.90 | 0.90 | 0.90 |
| | methicone) | | | | |
| A | Luvitol ® BL (BASF) (benzyl laurate) | 2.00 | 2.00 | 2.00 | 2.00 |
| A | Cetyl dimethicone | 2.00 | 2.00 | 2.00 | 2.00 |
| A | Polyglyceryl-4, isostearate, cetyl | 6.00 | 6.00 | 6.00 | 6.00 |
| | dimethicone copolyol and hexyl laurate | | | | |
| A | Cremophor ® GO 32 (BASF) (polyglyceryl-3 | 0.60 | 0.60 | 0.60 | 0.60 |
| | dioleate) | | | | |
| A | Glyceryl tribehenate | 3.00 | 3.00 | 3.00 | 3.00 |
| B | Masil ® SF V5 CG (BASF) (cyclomethicone) | 8.85 | 8.85 | 8.85 | 8.85 |
| B | Microfine zinc oxide | 2.00 | 2.00 | 2.00 | 2.00 |
| C | Deionized water | ad 100 | ad 100 | ad 100 | ad 100 |
| C | Xanthan gum NF | 0.10 | 0.10 | 0.10 | 0.10 |
| C | Disodium EDTA | 0.10 | 0.10 | 0.10 | 0.10 |
| C | Sodium chloride | 1.00 | 1.00 | 1.00 | 1.00 |
| C | Glycerin | 3.00 | 3.00 | 3.00 | 3.00 |
| C | Sclerotium Gum (such as in Tinocare ® GL) | 0.01%-1% | 0.01%-1% | 0.01%-1% | 0.01%-1% |
| D | Panthenol USP (BASF) (panthenol) | 0.10 | 0.10 | 0.10 | 0.10 |
| D | Sodium ascorbyl phosphate | 0.20 | 0.20 | 0.20 | 0.20 |
| E | Nanodispersion according to the invention; | 1.00 | 3.00 | 5.00 | 7.00 |
| | combination from1 to 8 | | | | |
| E | Vitamin A, C or E (free or derivative forms) | 0.80 | 0.50 | — | 1.00 |
| E | Fragrance | qs | qs | qs | qs |
| E | preservative (such as parabene) | 0.7 | 0.7 | 0.7 | 0.7 |

TABLE 3.7
CREAM FOUNDATION with Sunscreen
(Make up applications)

| | | a | b | c | d |
|---|---|---|---|---|---|
| Part | Ingredient | % b.w. | | | |
| A | Deionized Water | ad 100 | ad 100 | ad 100 | ad 100 |
| A | Magnesium Aluminum Silicate | 1.00 | 1.00 | 1.00 | 1.00 |
| A | Cellulose Gum | 0.20 | 0.20 | 0.20 | 0.20 |
| B | Deionized Water | 15.00 | 15.00 | 15.00 | 15.00 |
| B | Titanium Dioxide | 7.00 | 7.00 | 7.00 | 7.00 |
| B | Iron Oxides | 1.30 | 1.30 | 1.30 | 1.30 |
| B | Iron Oxides | 1.10 | 1.10 | 1.10 | 1.10 |
| B | Talc | 1.00 | 1.00 | 1.00 | 1.00 |
| B | Iron Oxides | 0.20 | 0.20 | 0.20 | 0.20 |
| C | Mineral Oil/Drakeol 7/Penreco | 8.00 | 8.00 | 8.00 | 8.00 |
| C | Glyceryl Stearate SE | 4.00 | 4.00 | 4.00 | 4.00 |
| C | Ethylhexyl Palmitate | 3.00 | 3.00 | 3.00 | 3.00 |
| C | Emulsifying Wax | 2.50 | 2.50 | 2.50 | 2.50 |
| C | Petrolatum/Snow Petrolatum/Penreco | 1.00 | 1.00 | 1.00 | 1.00 |
| D | Nanodispersion according to the invention; | 1.00 | 3.00 | 5.00 | 7.00 |
| | combination from1 to 8 | | | | |
| D | Vitamin A, C or E (free or derivative form) | 0.80 | 0.50 | — | 1.00 |
| D | Fragrance | qs | qs | qs | qs |

TABLE 3.8
Shower-gel

| Ingredients (amounts in % b.w.) | a | b | c | d |
|---|---|---|---|---|
| Sodium Laureth Sulfate | 15%-18% | 15%-18% | 15%-18% | 15%-18% |
| Lauryl Glucoside | 4%-6% | 4%-6% | 4%-6% | 4%-6% |
| Decyl Glucoside | 4%-6% | 4%-6% | 4%-6% | 4%-6% |
| Cocamidopropyl Betaine | 1%-5% | 1%-5% | 1%-5% | 1%-5% |
| Sodium Cocoamphoacetate | 0%-2% | 0%-2% | 0%-2% | 0%-2% |
| PEG-200 Glyceryl Palmate | 0%-2% | 0%-2% | 0%-2% | 0%-2% |
| PEG-150 | 4%-6% | 4%-6% | 4%-6% | 4%-6% |
| PEG-240 | 8%-10% | 8%-10% | 8%-10% | 8%-10% |
| PEG-220 | 5%-6% | 5%-6% | 5%-6% | 5%-6% |
| Triethyl Citrate | 2%-9% | 2%-9% | 2%-9% | 2%-9% |
| Glyceryl Mono Oleate | 1%-2% | 1%-2% | 1%-2% | 1%-2% |
| Decyl Oleate | 0%-2% | 0%-2% | 0%-2% | 0%-2% |
| Polyquaternium-7 | 0.05%-0.20% | 0.05%-0.20% | 0.05%-0.20% | 0.05%-0.20% |
| Potassium Citrate | 4%-12% | 4%-12% | 4%-12% | 4%-12% |
| Iminodisuccinate | 3%-5% | 3%-5% | 3%-5% | 3%-5% |
| PEG-40 Hydrogenated Castor Oil | 0%-2% | 0%-2% | 0%-2% | 0%-2% |
| Polyether-1 | 1%-4% | 1%-4% | 1%-4% | 1%-4% |
| Perfume/Fragrance | qs | qs | qs | qs |
| Preservative (such as parabene) | 0.7 | 0.7 | 0.7 | 0.7 |
| Water deionized | ad 100 | ad 100 | ad 100 | ad 100 |
| Nanodispersion according to the | 1.00% | 3.00% | 5.00% | 7.00% |
| invention; combination from1 to 8 | | | | |
| Vitamin A, C or E | 0.80% | 0.50% | — | 1.00% |
| (free or derivative form) | | | | |

TABLE 3.9
Clear shampoo

| Ingredients (amounts in % b.w.) | a | b | c | d |
|---|---|---|---|---|
| Sodium Lauryl Sulfate | 1% | 1% | 1% | 1% |
| Sodium Polyoxyethylene lauryl | 12% | 12% | 12% | 12% |
| sulfate | | | | |
| Lauroamidopropyl betaine | 1% | 1% | 1% | 1% |
| POE(16) lauryl ether | 1% | 1% | 1% | 1% |
| Glyceryl monoisodecyl ether | 0.5% | 0.5% | 0.5% | 0.5% |
| Cationized cellulose | 0.5% | 0.5% | 0.5% | 0.5% |
| Polyvinyl Alcohol | 0.2% | 0.2% | 0.2% | 0.2% |
| Glycerin | 10% | 10% | 10% | 10% |
| Tocopherol Acetate | 0.24% | 0.24% | 0.24% | 0.24% |
| Sodium Sulfate | 3% | 3% | 3% | 3% |
| Perfume/Fragrance | 0.1% | 0.1% | 0.1% | 0.1% |
| Sodium Hydroxide | ad pH 7 | ad pH 7 | ad pH 7 | ad pH 7 |
| Water deionized | ad 100 | ad 100 | ad 100 | ad 100 |
| Nanodispersion of the invention; | 1.00% | 3.00% | 5.00% | 7.00% |
| combination from 1 to 8 | | | | |
| Vitamin A, C or E | 0.80% | 0.50% | — | 1.00% |
| (free or derivative form) | | | | |

TABLE 3.10
Opaque silica-based dentifrice

| Ingredients | a | b | c | d |
|---|---|---|---|---|
| Sodium Lauryl Sulfate | 1.5-2.5% | 1.5-2.5% | 1.5-2.5% | 1.5-2.5% |
| Sodium Carboxymethyl | 0.7-1% | 0.7-1% | 0.7-1% | 0.7-1% |
| cellulose or | | | | |
| Xanthan gum | | | | |
| Sorbitol (70%) | 45% | 45% | 45% | 45% |
| Abrasive silica | 10% | 10% | 10% | 10% |
| Thickening silica | 8-10% | 8-10% | 8-10% | 8-10% |
| PEG 1500 | 5% | 5% | 5% | 5% |
| Titanium Dioxide | 1% | 1% | 1% | 1% |
| (opacifier) | | | | |
| Flavouring agents | <1.5% | <1.5% | <1.5% | <1.5% |
| (such as Sodium | | | | |
| Saccharinate) | | | | |
| Colorants | qs | qs | qs | qs |
| Preservative | <1% | <1% | <1% | <1% |
| Water deionized | ad 100 | ad 100 | ad 100 | ad 100 |
| Nanodispersion | 1.00% | 3.00% | 5.00% | 7.00% |
| according to the | | | | |
| invention; combination | | | | |
| from1 to 8 | | | | |
| Vitamin A, C or E | 0.80% | 0.50% | — | 1.00% |
| (free or derivative form) | | | | |

1. A cosmetic or pharmaceutical formulation containing an encapsulated antioxidant and a cosmetically or pharmaceutically acceptable carrier, where the antioxidant is selected from the group consisting of carbon bridged hindered phenols, ester bridged hindered phenols, amide bridged hindered phenols, lactones of hindered phenols, sterically hindered oxylamines and sterically hindered hydroxylamines.

2. A formulation of claim 1, containing a further active ingredient selected from the group consisting of oxidizable natural substances, vitamins, fragrances, and extracts from plants fungi, algae or animals.

3. A formulation of claim 2, wherein the active ingredient is coencapsulated together with the antioxidant.

4. A formulation according to claim 1 wherein the antioxidant is selected from the following compounds of formulae (1), (2) and/or (3) 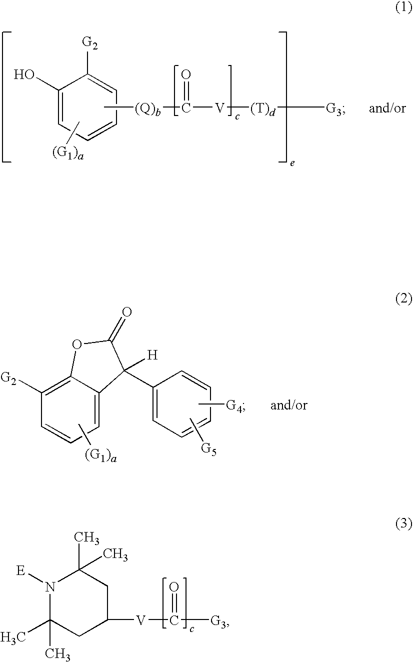
and acid addition salts thereof,
wherein in formulae (1), (2) and (3)
G₁ is hydrogen; C₁-C₂₂alkyl; C₁-C₂₂alkylthio; C₂-C₂₂alkylthioalkyl; C₅-C₇cycloalkyl; phenyl; C₇-C₉phenylalkyl; or SO₃M;
G₂ is C₁-C₂₂alkyl; C₅-C₇cycloalkyl; phenyl; or C₇-C₉phenylalkyl;
E is oxyl or hydroxyl;
Q is —CₘH₂ₘ—; 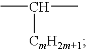
—CₘH₂ₘ—NH; a radical of formula 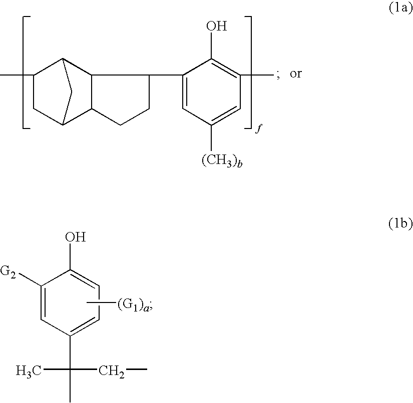
T is —CₙH₂ₙ—; —(CH₂)ₙ—O—CH₂—; phenylene; 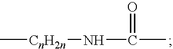
or a radical of formula 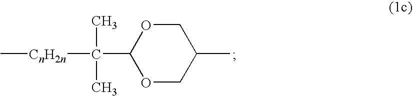
V is —O—; or —NH—;
a is 0; 1; or 2;
b, c and d and g are as defined in claim 1;
e is an integer from 1 to 4;
f is an integer from 1 to 3; and
m, n and p are each independently of one another an integer from 1 to 3;
q is 0 or an integer from 1 to 3;
if e=1, or in formula (3), then
G₃ is hydrogen; C₁-C₂₂alkyl; C₅-C₇cycloalkyl; C₁-C₂₂alkylthio; C₂-C₂₂alkylthioalkyl; C₂-C₁₈alkenyl; C₁-C₁₈phenylalkyl; M; SO₃M; a radical of formula 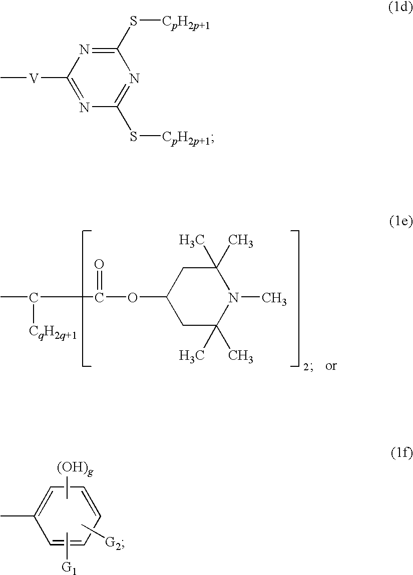
or G₃ is propyl substituted by OH and/or by C₂-C₂₂alkanoyloxy;
M is alkali; ammonium; H;
if e=2, then
G₃ is a direct bond; —CH₂—; 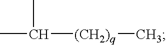
or —S—; or G₃ is propyl substituted by OH or C₂-C₂₂alkanoyloxy;
if
e=3, then
G₃ is the radical of formula 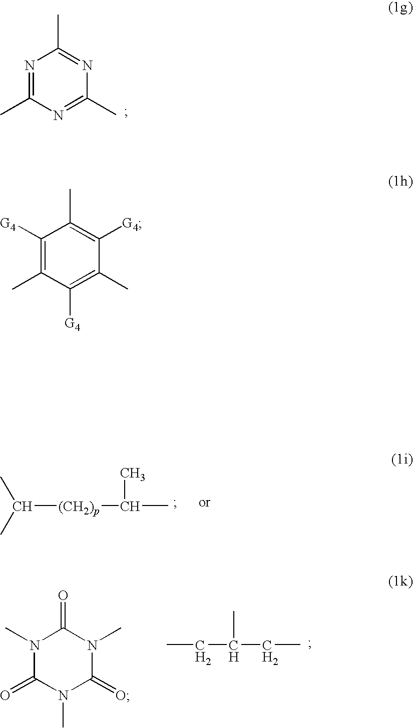
if
e=4, then
G₃ is 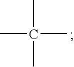
G₄ and G₅ are each independently of the other hydrogen; or C₁-C₂₂alkyl;
and the compounds of the formulae (16), (18), (20), (21), (22), or (23)

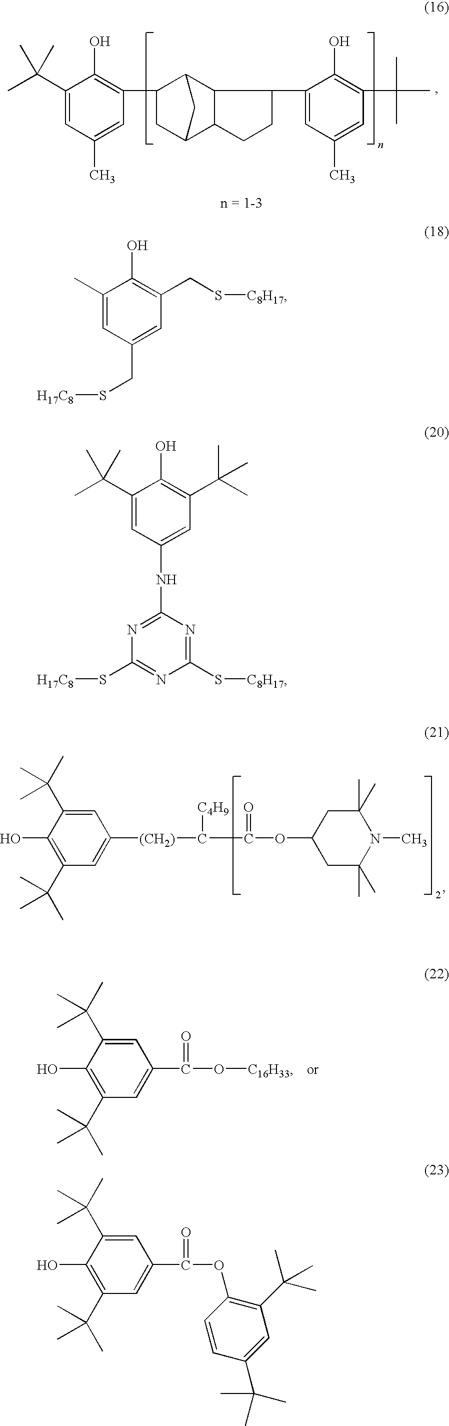

5. A formulation of claim 4, where in the compounds
a is 1;
Q, where present, is —CₘH₂ₘ— and, preferably, a methylene or ethylene radical,
T, where present, is —CₙH₂ₙ— or phenylene;
G₃ is hydrogen; C₁-C₂₂alkyl; SO₃M; propyl substituted by OH and/or by C₂-C₂₂alkanoyloxy;
or G₃ is a direct bond; —CH₂—; 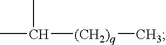
or propylene substituted by OH or C₂-C₂₂alkanoyloxy;
or G₃ is 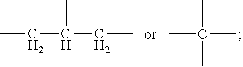
where the compound of the formula (3) conforms to the formula 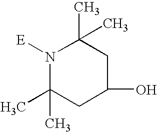
where E is oxyl or hydroxyl;
or is a cosmetically or pharmaceutically acceptable acid addition salt thereof;
especially where in the compound of the formula (1) or (2)
G₁ and G₂ are, independently of each other, C₁-C₅alkyl, especially tert.-butyl, and G₁ is located in meta-position relative to G₂; and
G₄ and G₅ independently are H or C₁-C₄alkyl, especially methyl.

6. A formulation of claim 4, where the encapsulated antioxidant is selected from compounds of the formula (1) or (2), where in the compound of formula (1) e is 2, 3 or 4.

7. A formulation of claim 4, wherein the compound (1), (2), (3) is selected from the compounds of formulae (7) to (35) 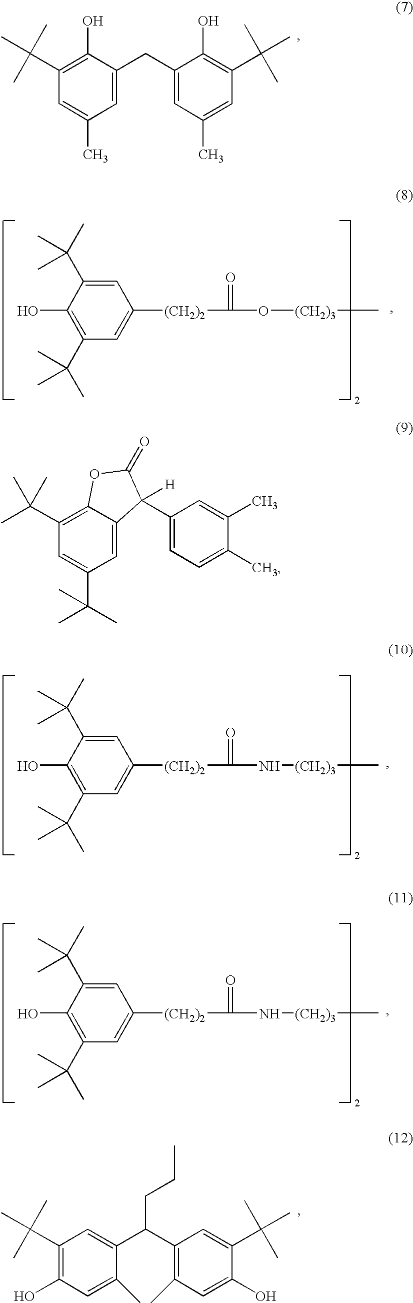 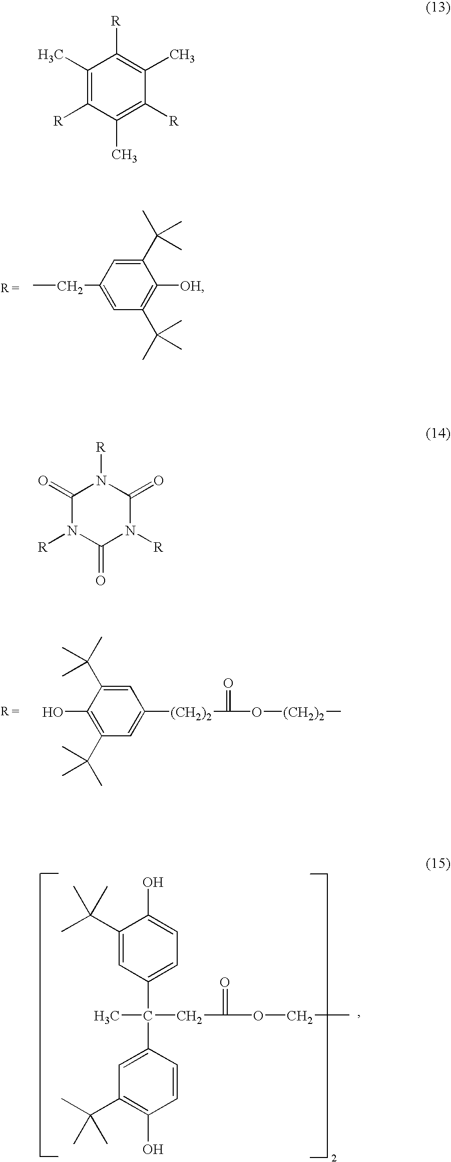 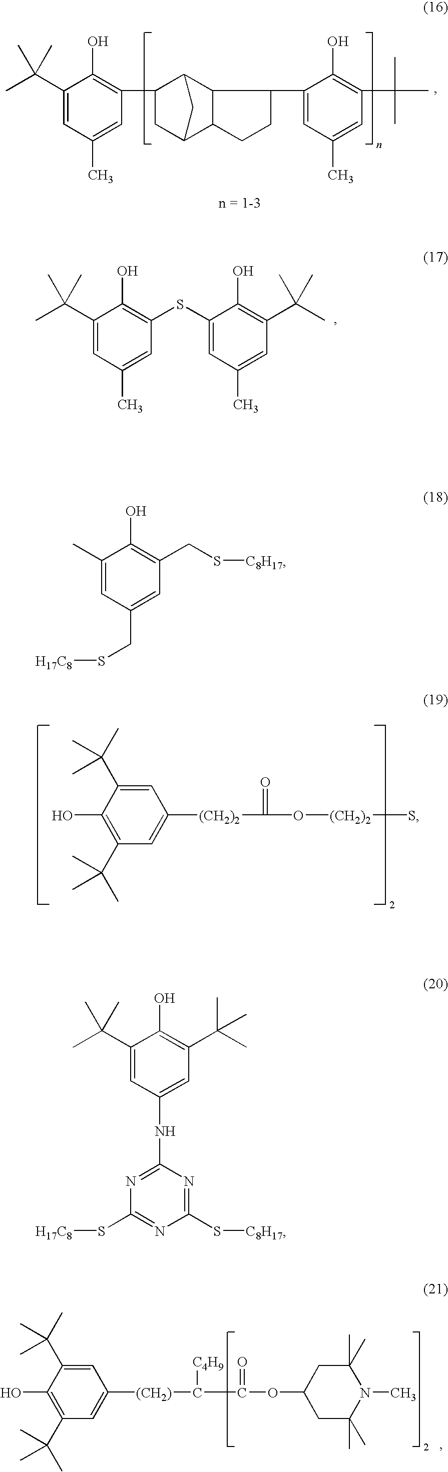 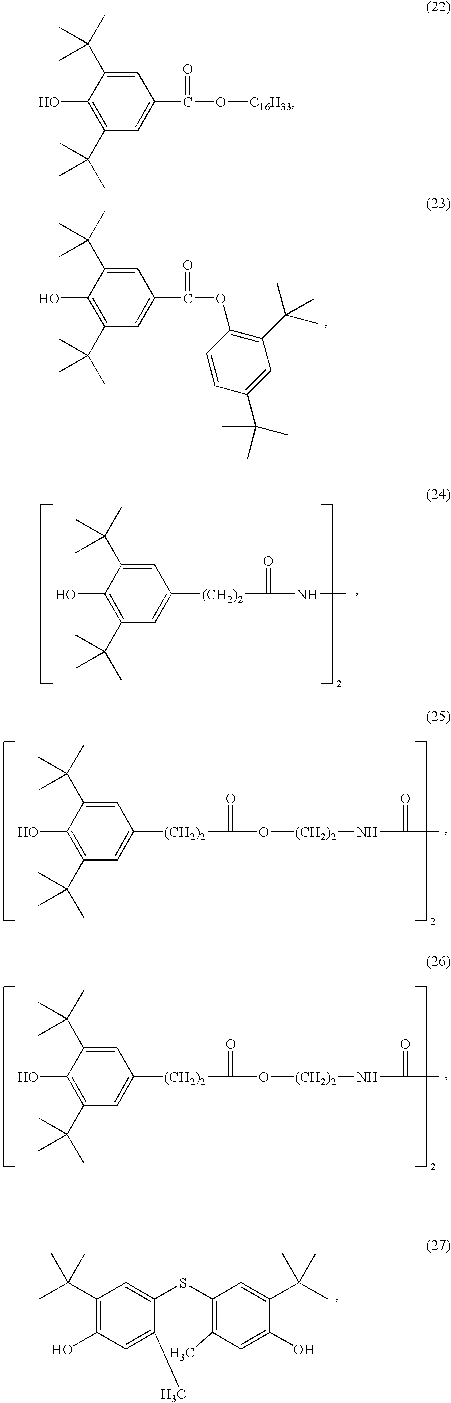 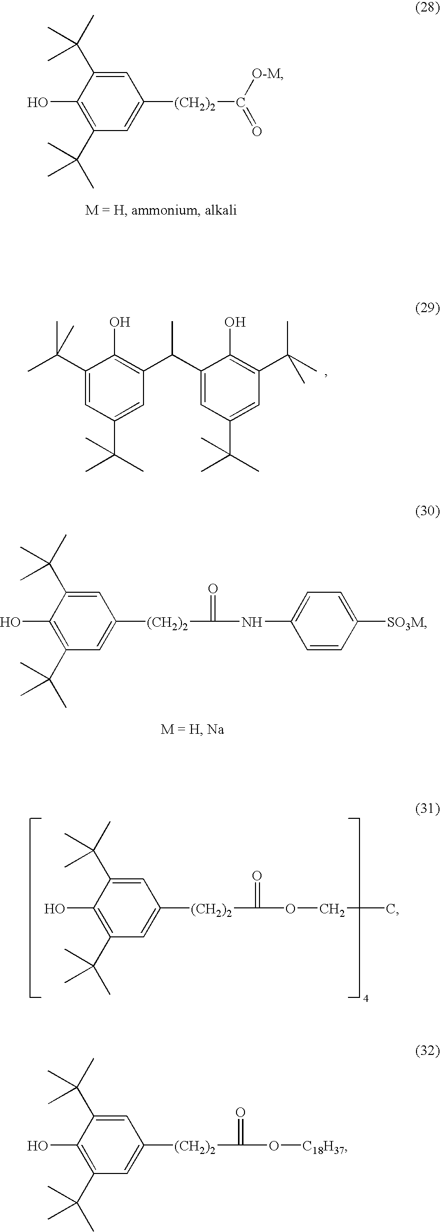 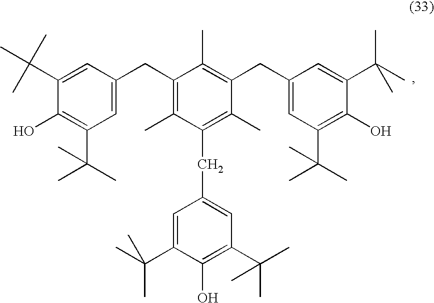
(34) the reaction product of glycerine, coconut oil and a compound of the

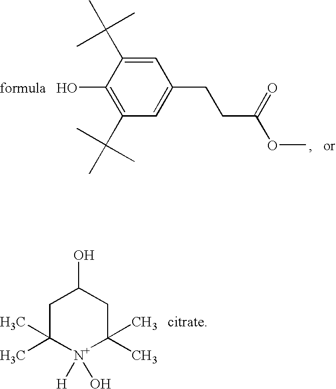

8. A formulation according to claim 1, which contains the encapsulated antioxidant in nanodispersed form, wherein the particles have a number average diameter smaller than 300 nm.

9. A formulation according to claim 1, wherein each of the antioxidant and the further active ingredient, if present, is contained in a total amount of 0.0001 to 10% by weight of the total composition.

10. A formulation according to claim 2, wherein the weight ratio of antioxidant to active ingredient ranges from 1:10 to 10:1.

11. A formulation according to claim 1, which additionally comprises at least one substance selected from the group consisting of antiphlogistic agents, antiinflammatory agents, vitamins, antipsoriatic agents, further skin actives, non-encapsulated antioxidants, cell proliferation regulators, antiallergic, UV protecting, moisturizing, antiaging agents, and DNA-protectants.

12. A method for the preparation of a formulation according to claim 1, wherein the encapsulation comprises the steps of mixing
(a) a membrane-forming molecule,
(b) a coemulsifier and
(c) a lipophilic component comprising the antioxidant and optionally a further active ingredient,
until a homogeneous clear liquid is obtained.

13. A method of claim 12, which comprises mixing of
(a) 5 to 20% by weight of a phospholipid,
(b) 15 to 40% by weight of a coemulsifier,
(c) 30 to 70% by weight of a lipophilic component comprising an antioxidant of formulae (1) to (3) and optionally a cosmetically acceptable lipid and/or an active ingredient selected from the group consisting of oxidizable natural substances, vitamins, plant extracts, and fragrances; and
(d) 0 to 30% by weight of an alcohol,
with the sum of percentages of the components (a), (b), (c) and (d) adding to 100%.

14. A method for the protection of an active ingredient in a cosmetic or pharmaceutical formulation from premature degradation by light, oxygen and/or heat said method comprises adding to said formulation an effective amount of an encapsulated antioxidant selected from the group consisting of carbon bridged hindered phenols, ester bridged hindered phenols, amide bridged hindered phenols, lactones of hindered phenols, sterically hindered oxylamines and sterically hindered hydroxylamines.

15. A method according to claim 14 wherein said cosmetic or pharmaceutical formulation is selected from the group consisting of skin care or skin delivery formulation, injectable solution, infusion solution, eye drop, drinking solution, dietary or enriched food, an oral care formulation, drinking gargles, in-halants, and food additive.

16. A method according to claim 15, where the antioxidant is coencapsulated together with a further active ingredient selected from the group consisting of oxidizable natural substance, vitamin, plant extract, and fragrance, for the local treatment or prevention of radical induced body impairments.

17. (canceled)

18. A formulation according to claim 2 wherein vitamins are selected from the group consisting vitamin A, vitamin C and vitamin E, in free or modified form.

19. A formulation according to claim 8, which contains the encapsulated antioxidant in nanodispersed form, wherein the particles have a number average diameter range of 10 to 80 nm.

20. A formulation according to claim 9, wherein each of the antioxidant and the further active ingredient, if present, is contained in a total amount of from 0.0005 to 5%, by weight of the total composition.

21. A method according to claim 12, wherein the encapsulation comprises the steps of mixing
(a) a membrane-forming molecule,
(b) a coemulsifier and
(c) a lipophilic component comprising the antioxidant and optionally a further active ingredient,
until a homogeneous clear, essentially anhydrous, liquid is obtained.

22. A method according to claim 16, where the antioxidant is coencapsulated together with a further active ingredient selected from the group consisting of oxidizable natural substance, vitamin, plant extract, and fragrance, for the preparation of a cosmetic or pharmaceutical formulation for the local treatment of inflammatory and allergic conditions of the skin.
